(19) United States
(12) Patent Application Publication
(10) Pub. No.: US 2025/0388937 A1
(43) Pub. Date: Dec. 25, 2025

(54) Low Energy Production Process for Renewable Hydrocarbons

(71) Applicant: Gevo, Inc., Englewood, CO (US)

(72) Inventors: Patrick Gruber, Englewood, CO (US); Christopher Ryan, Englewood, CO (US); Adrian Galvez, III, Englewood, CO (US); Paul Starkey, Englewood, CO (US)

(21) Appl. No.: 19/314,939

(22) Filed: Aug. 29, 2025

Related U.S. Application Data
(63) Continuation of application No. PCT/US24/19976, filed on Mar. 14, 2024.
(60) Provisional application No. 63/490,629, filed on Mar. 16, 2023.

(51) Int. Cl. C12P 7/10; C10G 3/00; C10G 65/02

(52) U.S. Cl. CPC C12P 7/10; C10G 3/00; C10G 65/02; C10G 2400/04; C10G 2400/08

(57) ABSTRACT

Disclosed is a carbon neutral, zero net energy process, system, and plant for converting lower linear and branched mono-olefins, derived from C₂-C₅ bio-based alcohols to higher hydrocarbons, to one or more C₈-C₂₄ hydrocarbons. Also disclosed are a carbon neutral, zero net energy process for oligomerization of branched and/or linear C₃-C₈ olefins to renewable diesel fuel and/or jet fuel.

### RELATED APPLICATIONS

The disclosure claims priority to U.S. Provisional Patent Application Ser. No. 63/490,629, titled Low Energy Production Process for Renewable Hydrocarbons, filed 16 Mar. 2023, the disclosure of which is incorporated herein by reference as if set out in full.

### FIELD

This disclosure relates to systems and methods for producing renewable alcohols and hydrocarbons. The present disclosure also relates to a carbon neutral, zero net energy process for converting one or more C₂-C₅ alcohols to one or more C₈-C₂₄ hydrocarbons.

### BACKGROUND

Petroleum is a nonrenewable resource and its combustion results in carbon being released into the environment. There is an increasing demand for the use of biomass sources for replacing petroleum as the starting point for the synthesis of fuels. With the increased availability and reduced cost of biomass-derived alcohols, biomass-derived alcohols are inexpensive and renewable feedstock for making a variety of olefins for use in producing downstream hydrocarbons. As used in the present application, renewable hydrocarbons includes any hydrocarbon production that is wholly or partially based on a renewable sources, such as the aforementioned biomasses, as well as blended hydrocarbons, such as, for example, sustainable aviation fuel (“SAF”).

Current biogas production and spent grain processing technologies produce waste products, such as proteins, that could be used to produce other products, e.g. animal feed, and thus are not fully efficient technologies. Furthermore, current technologies fail to address the issues created by high nitrogen concentrations in anaerobic digestion systems. Current integrated systems and processes for improving yield and efficiency of alcohol production also suffer from inefficiencies and limitations. For example, there is a need for systems that produce stillage specifications that have fully efficient and low cost anaerobic digestion. There also is a need for production of high energy density fuels, such as hydrocarbon fuels or transportation fuels (including SAF), especially those with a no or low carbon footprint (i.e. carbon neutral).

Current technologies employing petroleum fuels inherently have a high carbon footprint, in part, because they are produced by recovering oil from the earth, which ultimately ends up in the atmosphere once the fuel is combusted, such as in an engine. Use of biodiesel can pose a problem as well, because it is not a hydrocarbon and its chemical properties make its fuel properties inferior and its blending limits low. Renewable diesel is a hydrocarbon that is commonly used, but it is produced from fats, greases, and animal- and plant-derived oils that are in relatively low supply considering the high global demand for transportation fuels. Additionally, the coproducts produced from sources like renewable diesel and propane have a low octane value and high vapor pressure, and therefore very little value for recycling into transportation fuel markets, such as, the gasoline market or SAF market, to reduce net carbon footprint or net energy use.

Today there is a demand for zero or low carbon (i.e. carbon neutral) and negative carbon (i.e. carbon sequestering) footprints, for high energy density transportation fuels such as jet fuel (SAF), diesel fuel, bunker fuel, or gasoline (generically “fuels”). These fuels can be used in existing assets and blended at high levels with conventional fuels. In some cases, the fuels do not require blending. Conventional fuels, as used in this application, generally refers to fuels derived from hydrocarbons recovered from hydrocarbon deposits worldwide. Some present day blended fuels, such as ethanol blended with gasoline, have low energy density and blend limits due to incompatibility with existing assets. Moreover, the ethanol production assets currently existing were not created to be carbon neutral. Renewable diesel is an example of a high energy carbon fuel, but the carbon footprint is much higher than zero. Moreover, renewable diesel presently has a limited market reach, mostly limited to the diesel fuel market, and the amounts of renewable feedstocks for production of renewable diesel are low compared to the market size and demand.

To date, the necessary steps to deliver a commercially viable carbon neutral transportation fuel with high energy density made from carbohydrates has not been identified. As can be seen in the California Low Carbon Fuel System pathway reports, there are no viable carbohydrate based fuels with high energy density (i.e. greater than 110,000 BTU/gallon) nor hydrocarbon fuels with zero carbon score.

Current commercial ethanol processes rely on distillation and evaporation as fermentation product recovery unit operations, which require large amounts of energy, even when heat is integrated to minimize energy. For example, according to the Renewable Fuels Association (RFA) in a 2016 publication, a typical ethanol plant uses approximately 26,700 BTU/gallon of process energy (thermal energy plus electrical energy) from outside energy sources (hereafter referred to as “external energy”). External energy as used herein refers to energy sources brought in from outside ethanol plant limits. Ethanol has a low energy density of 76,300 BTU/gallon, which means production process energy represents approximately 35% of the output energy. This process energy, natural gas and grid electricity, represent a large carbon footprint and therefore prevent the processes from achieving carbon neutrality. Moreover, ethanol has an energy density approximately 65% of that of petroleum gasoline (116,000 BTU/gallon). Ethanol therefore poses a limit on the transportation range in a vehicle, and ethanol cannot be used in jet fuel, diesel fuel, nor bunker fuel. Furthermore, its blend ratio in gasoline is limited.

Achieving a carbon neutral process would require a departure from typical fermentation production designs, exemplified by bio-based ethanol production. Typical production designs have been optimized for managing capital and operating costs when energy costs are very low, and when there is not an impetus to reduce carbon footprint. However, based on current global liquid fuels and chemical production, there will be an ever increasing desire to reduce carbon footprint and energy use.

Traditional stillage anaerobic digestion (AD) systems come in various designs. The designs that are most efficient and lower cost typically require lower total nitrogen, lower chemical oxygen demand (COD), and lower suspended solids. The total nitrogen limit helps avoid pH levels and ammonia levels that could destroy the microorganisms in the AD system that convert various components into methane. This is not compatible with traditional stillage compositions in the industry today.

Waste carbohydrates have a low carbon footprint because they are wastes, however, there is not enough supply of waste carbohydrates to meet the current need for transportation fuels. Carbohydrate based feedstocks that are in high abundance, such as sugar cane, corn starch, wheat starch, sugar beets, and cassava, typically are not low carbon footprint due to growing practices used for those materials and/or the inability to identify and track feedstocks grown with sustainable practices through the supply chain.

Alcohol to hydrocarbon fuels conversion processes have not been commercially deployed for various reasons. The desire for renewable fuels with high energy density has only been a recent occurrence. The ability to convert alcohols to higher energy densit fuels, such as transportation fuels that meet fuel specifications, which requires control over the molecular architecture of the products, has not been developed. Therefore, there has been an unmet need for the supply of renewable alcohols having a carbon footprint low enough to enable the resultant hydrocarbon to have an attractive carbon footprint.

Provided herein are, inter alia, solutions to the above discussed and other problems in the field.

### SUMMARY

This disclosure describes, among other things, a system for producing renewable hydrocarbons. The system includes a fractionation subsystem for processing a biomass that contains a carbohydrate; a fermentation subsystem for converting the carbohydrate to a fermentation product; a water treatment subsystem for receiving a first portion of the fermentation product; an alcohol enrichment subsystem for receiving a second portion of the fermentation product; a hydrocarbon production subsystem for receiving an alcohol and producing the renewable hydrocarbons; and an energy management subsystem for receiving fuel stream from the system and for delivering power to the system.

In some embodiments, one or more of the following features may be included in any feasible combination. For example, the fractionation subsystem may include at least one of: a storage vessel for storing the biomass; a pulverizer for breaking apart the biomass; a pretreatment vessel for pretreating the biomass; a treatment vessel for treating the biomass to produce the carbohydrate; and a high-temperature-short-time (HTST) vessel for pasteurizing the carbohydrate.

In some embodiments, the fermentation subsystem may include at least one of: a fermenter for converting the carbohydrate to the fermentation product; a nutrient addition subsystem; a pH adjustment subsystem; and an inoculum propagation subsystem.

In some embodiments, the water treatment subsystem may include at least one of: a beer well for receiving the first portion of the fermentation product; a microorganism separation device for removing microorganisms from the first portion of the fermentation product; a first distillation column for separating the first portion of the fermentation product into an alcohol and a bottom product; a digester for receiving a portion of the bottom product and for producing a biogas; and a biogas removal subsystem for removing a pollutant from the biogas.

In some embodiments, the alcohol enrichment subsystem may include at least one of: a flash tank for separating a condensate from the second portion of the fermentation product; a separation vessel for separating the condensate into a light phase and a heavy phase; an ion exchange vessel for purifying the light phase; a membrane separator for separating the light phase into an alcohol-high retentate and a water-rich permeate; a second distillation column for separating water from the alcohol-high retentate; and an alcohol storage vessel.

In some embodiments, the hydrocarbon production subsystem may include at least one of: a denitrogination subsystem for separating nitrogen from the alcohol; a dehydration subsystem for converting the alcohol to an olefin; a hydrocarbon pretreatment subsystem for conditioning an olefin feed; a hydrogen supply subsystem for providing hydrogen; and a hydrocarbon processing subsystem for converting the olefin feed to at least one of an iso-octane fraction, a C₁₂ alkane fraction, and/or C₁₆ alkane fraction.

In some embodiments, the hydrocarbon processing subsystem may include at least one of: a first reactor; a debutanizer; a second reactor; a separator; a first splitter; and/or a second splitter.

In some embodiments, the energy management subsystem may include at least one of: a fuel gas system for distributing fuel gas received from the hydrocarbon processing subsystem; a low pressure boiler for generating steam; a high pressure boiler for generating steam; a combined heat and power unit for generating steam and electricity; a wind turbine for generating electricity; an electric boiler for receiving renewable electricity and for heating water to produce steam; a biomass boiler for combusting biomass for heating water to produce steam; and/or a steam turbine for generating electricity.

In some embodiments, the biomass is corn, wheat, and/or sorghum. In some embodiments, the biomass has a negative carbon footprint. In some embodiments, the biomass is grown using strip till or no till. In some embodiments, the pulverizer is a mill. In some embodiments, the biomass is grown using agricultural practices to increase soil organic carbon. In some embodiments, the pretreatment vessel may include a recycled water input. In some embodiments, pretreating the biomass may include pretreating the biomass with an enzyme. In some embodiments, the treatment vessel may include a non-fermentable solids output. In some embodiments, the non-fermentable solids output may include dried distillers grain output and/or corn oil output. In some embodiments, a portion of the corn oil output may be used as fuel for a boiler.

In some embodiments, the fermenter is a continuous fermenter. In some embodiments, the fermentation product may include an alcohol. In some embodiments, the alcohol is isobutanol.

In some embodiments, the inoculum propagation subsystem is configured to provide a microorganism to the fermenter. In some embodiments, the microorganism is yeast. In some embodiments, the first portion of the fermentation product may include an alcohol. In some embodiments, the first portion of the fermentation product may include isobutanol. In some embodiments, the first portion of the fermentation product may include water. In some embodiments, the microorganism separation device may include a centrifuge. In some embodiments, the microorganism separation device may include a filter. In some embodiments, the microorganism separation device may include a settling tank.

In one aspect of the present disclosure, the first distillation column includes at least one vapor recompression subsystem. In another aspect of the present disclosure, the first distillation column includes at least one mechanical vapor recompression (MVR). In another aspect of the present disclosure, the first distillation column includes at least one thermal vapor recompression (TVR).

In one aspect of the present disclosure, the bottom product may include stillage. In some embodiments, the digester is an anaerobic digester. In another aspect of the present disclosure, the digester is a continuous digester. In another aspect of the present disclosure, the biogas removal subsystem may include a scrubber. In another aspect of the present disclosure, the pollutant is hydrogen sulfide.

In some embodiments, the flash tank receives the second portion of the fermentation product from the fermentation subsystem and returns part of the second portion of the fermentation product back to the fermentation subsystem. In some embodiments, the second portion of the fermentation product is a broth and an isobutanol lean broth is returned back to the fermentation subsystem. In some embodiments, the flash tank operates at atmospheric pressure. In some embodiments, the flash tank operates under a vacuum at a reduced pressure below atmospheric pressure. In some embodiments, the flash tank operates at a temperature below a temperature of the second portion of the fermentation product received from the fermentation subsystem. In some embodiments, the separation vessel is a liquid/liquid separator for separating the condensate into the light phase and the heavy phase. In some embodiments, the light phase contains a greater amount of alcohol than the heavy phase. In some embodiments, the alcohol is isobutanol and the liquid/liquid separator form a biphasic system. In some embodiments, the ion exchange vessel contains an ion exchange resin and receives the light phase received from the separation vessel and removes impurities from the light phase by trapping ions in the ion exchange resin. In some embodiments, the ion exchange vessel receives a caustic solution to regenerate the ion exchange resin by flushing the impurities to a beer well.

In some embodiments, the hydrocarbon production subsystem further includes at least one of: an oil and water separator for separating wastewater from the hydrocarbon processing subsystem; and a hydrocarbon storage vessel for storing at least one of iso-octane, C₁₂ blend, or C₁₆ blend from the hydrocarbon processing subsystem.

In some embodiments, the denitrogenation subsystem receives the alcohol from the alcohol enrichment subsystem. In some embodiments, the alcohol is a fuel grade isobutanol. In some embodiments, the dehydration subsystem receives the alcohol from the denitrogination subsystem and sends water and/or purge products to a beer well. In some embodiments, the olefin is a four carbon olefin.

In some embodiments, the hydrocarbon pretreatment subsystem may include at least one of: a coalescer for receiving the olefin from the dehydration subsystem; an adsorber for removing unreacted alcohols, water, and nitrogen; and a feed receiver.

In some embodiments, the coalescer further receives wash water and sends waste water to a surge tank and/or a dehydration unit. In some embodiments, the coalescer further receives wash water and sends waste water to a surge tank and/or a dehydration unit. In some embodiments, the olefin is a C₄ olefin. In some embodiments, the unreacted alcohol is isobutanol.

In some embodiments, the feed receiver receives the olefin from the adsorber. In some embodiments, the first reactor is a solid acid catalyzed oligomerization (e.g. Polynaptha™ as one commercial example) reactor. In some embodiments, the first reactor receives the olefin from the feed receiver and oligomerizes the olefin to a polyolefin. In some embodiments, the debutanizer receives a polyolefin from the first reactor. In some embodiments, the debutanizer recycles an unreacted olefin and/or paraffin to the first reactor. In some embodiments, the second reactor is a hydrogenation reactor. In some embodiments, the second reactor receives a polyolefin from the debutanizer. In some embodiments, the second reactor further receives hydrogen from a hydrogen supply subsystem and hydrogenates the polyolefin to a C₈-C₁₆ alkane. In some embodiments, the separator removes an unreacted olefin and/or paraffin from the C₈-C₁₆ alkane. In some embodiments, the unreacted olefins and/or paraffins include C₄ products. In some embodiments, the separator recycles a portion of the C₈-C₁₆ alkane to the second reactor. In some embodiments, the first splitter separates the isooctane fraction from the C₈-C₁₆ alkane.

In some embodiments, the isooctane fraction may include about 85%, 90%, 91%, 92%, 93%, 94%, 95, 96%, 97%, 98%, 99% or 100% wt % C₈ alkanes, about 1%, 2%, 3%, 4% or 5 vol % olefins, and/or about 2 ppm, 4 ppm, 6 ppm, 8 ppm or 10 ppm sulfur.

In other embodiments, the isooctane fraction may include greater than 95 wt % C₈ alkanes, less than 5 vol % olefins, and/or less than 10 ppm sulfur. In some embodiments, the first splitter separates an unreacted olefin and/or paraffin from the C₈-C₁₆ alkane. In some embodiments, the unreacted olefins and/or paraffins include C₄ products. In some embodiments, the second splitter separates the C₁₂ alkane fraction from the C₁₈ fraction.

In some embodiments, the fuel gas system provides fuel gas to the low pressure boiler. In some embodiments, the fuel gas system provides fuel gas to the high pressure boiler. In some embodiments, the fuel gas system provides fuel gas to a fired heater. In some embodiments, the low pressure boiler is further configured to received natural gas and/or biogas from an anaerobic digester. In some embodiments, the combined heat and power unit is further configured to received natural gas and/or biogas from an anaerobic digester. In some embodiments, the low pressure boiler in the fuel gas system is configured to receive corn oil, biomass, or products from the pyrolysis of biomass as low carbon intensity fuels.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter of this disclosure are contemplated as being part of the embodiments disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

FIG. 1 is a flow chart illustrating a low energy production process consistent with embodiments of the current disclosure.

FIG. 2 is a flow chart illustrating a process for conversion of carbohydrates to hydrocarbons consistent with embodiments of the current disclosure.

FIG. 3 is a flow chart illustrating an isobutanol production process consistent with embodiments of the current disclosure.

FIG. 4 is a flow chart illustrating a carbon neutral, zero net energy isobutanol process consistent with embodiments of the current disclosure.

FIG. 5 is a flow chart illustrating a continuation of the process of FIG. 4.

FIG. 6 is a flow chart illustrating a hydrocarbon pretreatment and processing system consistent with embodiments of the current disclosure.

FIG. 7 is a flow chart illustrating an ethanol pretreatment and processing system consistent with embodiments of the current disclosure.

FIG. 8 is a diagram illustrating the integration of alcohol to hydrocarbons waste heat with biomass processing.

FIG. 9 is a process flow diagram illustrating a typical ethanol distillation system.

FIG. 10 is a process flow diagram illustrating a conventional triple-effect evaporator used in ethanol purification.

FIG. 11 is a process flow diagram illustrating the use of multiple MVR fans in ethanol purification.

FIG. 12 is a diagram illustrating the evaporator system integrated with FIG. 11.

FIG. 13 is a graph showing a comparison of carbon intensities for different petroleum-based fuels as well as the carbon intensity reductions by employing various aspects of the low energy process to produce biofuels.

FIG. 14 illustrates the impact of agricultural practice on total life-cycle greenhouse gas (GHG) emissions of sustainable aviation fuel (SAF).

### DETAILED DESCRIPTION

Disclosed herein is a process for producing high energy density, zero carbon footprint hydrocarbon fuels, using a feedstock that is in large supply throughout the world and can be procured with zero carbon footprint itself: carbohydrates. In the systems and processes described herein, carbohydrates (e.g., corn, wheat, and/or sorghum) are converted to hydrocarbons. In general, the systems and processes achieve a net zero energy use and zero carbon footprint by using a low carbon footprint carbohydrate-based feedstock, a feedstock fractionation process which can be employed prior to or post-fermentation, to remove most protein and water-insoluble solids, a fermentation process, a biogas generation process using fermentation stillage, and an alcohol-to-hydrocarbon conversion process.

In the systems and processes disclosed herein, the total external process energy requirements are less than about 20,000 BTU/gallon, 15,000 BTU/gallon, 10,000 BTU/gallon, 8,000 BTU/gallon, 6,000 BTU/gallon, 4,000 BTU/gallon, 2,000 BTU/gallon; and the product is a hydrocarbon having an energy density of at least 100,000 BTU/gallon, 105,000 BTU/gallon, 110,000 BTU/gallon, 115,000 BTU/gallon, 120,000 BTU/gallon, 125,000 BTU/gallon, 130,000 BTU/gallon, 135,000 BTU/gallon, 140,000 BTU/gallon, 145,000 BTU/gallon, or 150,000 BTU/gallon. Therefore the external process energy represents no more than 6%, 8%, 10%, or 12% of the product energy. Achieving a zero carbon footprint, i.e. carbon neutrality, is possible due to this low external process energy requirement plus proper selection of the carbohydrate feedstock. Achieving this for ethanol or any other low energy density fuel is easier than for other fuels, given the lower energy density of the low energy density fuel. However, demand exists for a fuel that has an energy density closer to that of petroleum fuels, for example energy density of at least 65%, 70%, 75%, 80% or 85% of petroleum gasoline or jet fuel.

In the hydrocarbon processes described herein, any gasoline distillation range hydrocarbon products provides an octane rating of at least 50, 55, 60, 65, 70, 75 or 80 to ensure economical blending with other components to produce gasoline. This contrasts renewable diesel production today, which gives a coproduct (propane) with an octane value less than 50, 55, 60, 65, 70, 75 or 80.

In the processes described herein, carbon intensity (CI) (gCO2e/MJ) is used as a measure of a high energy density carbohydrate derived fuel using reduced tillage corn feedstock, stillage biogas, and wind power.

Described and illustrated herein are carbon neutral, zero net energy systems, equipment, processes, features, and functions to address the issues in current systems. In the following description, certain specific details are set forth in order to provide a thorough understanding of various embodiments. In some instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

Reference throughout this specification to “some embodiments”, “one embodiment” or “an embodiment” means a particular feature, structure or characteristic described in connection with the embodiment is included in at least one embodiment. Thus, the appearances of the phrases “in some embodiments”, “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The word “about” when immediately preceding a numerical value means a range of plus or minus 10% of that value, e.g., “about 50” means 45 to 55, “about 25,000” means 22,500 to 27,500, etc. Furthermore, the phrases “less than about” a value or “greater than about” a value should be understood in view of the definition of the term “about” provided herein.

The “Carbon Intensity” (CI), as described herein, is calculated based on industry standard Argonne National Laboratory's (Argonne) Greenhouse gases, Regulated Emissions, and Energy use in Technologies (GREET®) Model. However, other CI frameworks, although not used for calculating the CI values in this application, also can be used, including but not limited to: The California Low-Carbon Fuel Standard (CA-GREET3.0), The Carbon Offsetting and Reduction Scheme for International Aviation by the UN International Civil Aviation Organization (ICAO CORSIA), The Environmental Protection Agency's Renewable Fuel Standard (EPA RFS), The European Renewable Energy Directive (EU REDII), The Canadian Clean Fuel Standard (CFS), and a carbon reduction regulation used by Brazil (RenovaBio). Argonne's GREET® Model was developed by Argonne with the support of the U.S. Department of Energy (DOE), to consider carbon intensity related to the energy and environmental effects of fuels and vehicle technologies. Argonne GREET® model can be applied to all aspects of the fuel supply chain, enabling a true apples-to-apples comparison of greenhouse gas emissions for suppliers, consumers, industry, and regulators. Argonne GREET® Model quantifies emissions over the entire life cycle, and is deemed superior to other life-cycle analysis (LCA) tools available to date. Argonne GREET® Model includes the Feedstock Carbon Intensity Calculator (FD-CIC) and the Carbon Calculator for Land Use Change from Biofuels (CCLUB). These models can be downloaded from https://greet.es.anl.gov/.

The C₁₂₊ oligomers can be utilized directly for the production of renewable diesel fuel and renewable jet fuel post hydrogenation. For example, for the isobutanol/iC₄ process.

### Process Scheme for a Low Energy Production Process for Producing Biofuels

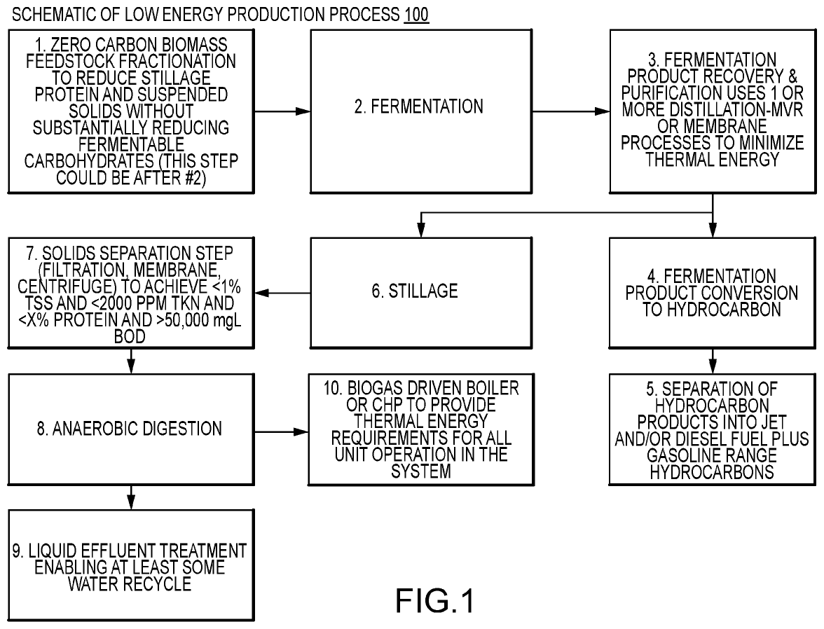

A schematic of a low energy production process 100 is shown in FIG. 1. The low energy production process 100 is sometimes referred to as a net zero carbon footprint process (sometimes “Net Zero” or “NZ”). Regarding the feedstock and feedstock selection, it is now known that certain growing practices allow for carbohydrates having zero carbon footprint or lower. For example, reduce tillage corn grown with reasonable yields and no or low irrigation has been estimated to have a low or zero carbon footprint.

Referring to Step 1 of FIG. 1, the low energy production process 100 begins with preparing a fermentation feedstock. The fermentation feedstock is a biomass source that contains carbohydrates and, optionally, reducing water insoluble solids and proteins once the feedstock is introduced to water to make a slurry. It has been determined that a carbohydrate source (e.g., a high sugar corn) can impact the ability to ultimately meet a net zero energy, carbon neutral target. As mentioned in the FIG. 1, the feedstock is fractionated as needed for the process.

Step 2 includes conducting a fermentation to convert the carbohydrate to fermentation product. Alternatively, Step 1 can be carried out after Step 2 process.

Step 3 includes subjecting the fermentation broth containing a fermentation product to one or more fermentation product separation processes, which produces a first stream that contains the fermentation product and a second stream consisting of fermentation spent broth. The fermentation product is purified such that it can meet product specifications using one or more energy efficient processes consistent with the technology described in the present application. Optionally, the fermentation product is subjected to one or more chemical transformations that result in one or more hydrocarbons that, in certain embodiments described herein, meet specifications for use in fuels and/or chemicals. A low thermal energy demanding unit operates to recover the ferementation product including, as described by the present application, distillation processes and equipment fitted with mechanical vapor recompression (MVR) and/or membranes, filters, or centrifuges, which enable an overall production process thermal energy that is less than the zero carbon energy generated by the fuel gas system.

In step 4, the fermentation product is converted to a hydrocarbon product having an energy density of at least 100,000 BTU/gallon, 105,000 BTU/gallon, 110,000 BTU/gallon, 115,000 BTU/gallon, 120,000 BTU/gallon, 125,000 BTU/gallon, 130,000 BTU/gallon, 135,000 BTU/gallon, 140,000 BTU/gallon, 145,000 BTU/gallon, or 150,000 BTU/gallon using a combination of dehydration and oligomerization followed by hydrogenation. The heat output of the oligomerization step can be integrated with the first conversion step, which includes dehydration, such that the overall net energy required to convert alcohol to hydrocarbon is minimized to achieve a low energy process.

In step 5, the hydrocarbon product is separated into jet fuel, diesel fuel, bunker fuel, and/or gasoline distillation range hydrocarbons. The hydrocarbon product, produced from the fermented product, generally consists of a range of C₆ to C₂₀ hydrocarbons that are useful for transportation fuel.

At step 6, the stillage has a composition which is substantially free of proteins and suspended solids, thereby allowing for a water treatment system that is efficient and low cost. Specifically, the stillage composition may, in certain embodiments, have the following characteristics: greater than 40,000 chemical oxygen demand (COD), 45,000 COD, 50,000 COD, 55,000 COD, 60,000 COD, 65,000 COD, 70,000 COD, 75,000 COD, 80,000 COD, 85,000 COD, or 90,000 COD; less than 0.2%, 0.4%, 0.8%, 1%, 1.2%, or 1.4% Total Suspended Solids (TSS); less than 1000 ppm, 1300 ppm, 1600 ppm, 1900 ppm, 2200 ppm, or 2500 ppm Total Kjeldahl Nitrogen (TKN); less than 2000 ppm, 4000 ppm, 6000 ppm, 8000 ppm, 10,000 ppm, or 12,000 ppm protein.

Step 7 may include subjecting the stillage to one or more filtration processes to reduce the suspended solids and proteins, such as to achieve these specifications. In certain embodiments, step 7 may be optional depending on the compositions.

In step 8, the fermentation spent broth is subjected to an anaerobic digestion process that, in certain embodiments, produces at least 6,000 BTU, 8,000 BTU, 10,000 BTU, 12,000 BTU, 18,000 BTU, 22, 000 BTU, 25,000 BTU, 30,000 BTU, 35,000 BTU, or 40,000 BTU of methane per gallon of hydrocarbon product. The BTUs of methane recovered represents, in certain aspects, at least 6%, 8%, 10%, 12%, 18%, 22%, 25%, 30%, 35%, or 40% of the energy content of the product being produced. In some embodiments, the anaerobic digestion process can produce at least 20,000 BTU of methane per gallon of hydrocarbon product, which can represent at least 20% of the energy content of the product being produced. In some embodiments, the amount of methane generated is at least as great as the external process energy required. As an alternative to using the methane produced in step 8 as an energy source, at least some of the oils produced during biomass fractionation (Step 1) may be used as an energy source to drive the entire production process, or at least a portion of the production process, such that less external energy sources are required, which reduces the overall carbon footprint, at least in part.

Subsequent use of at least some of this methane as an energy source to drive the production process, whether all or part of the production process, results in the need for less external energy sources. For example, with a COD of 82,000 mg/L in a 58 mmgpy isobutanol plant, there would be approximately 233 MMBTU/hr, or 500 MMBTU/day, of methane generated in an anaerobic digester. This equates to approx. 2 million MMBTU/yr, assuming 24 hours per day and 350 days per year. The overall process thermal energy required is ˜3300 MMBTU/day, not including energy required to dry any of the solids streams, and approximately 5500 MMBTU/day if 90% of the solids are dried to approx. 88% dry matter, with the rest being supplied to the feed market on a wet basis.

At step 9, a water treatment processes produces water. The recovered water can be recycled back to the front end of the process.

At step 10, one or more boilers can accept the gas outlet of the anaerobic digester (AD) system. The heat or energy from the AD system, in certain embodiments, produces steam that drives some of process. Using the heat/energy from the AD system also serves to lower overall process external energy requirements.

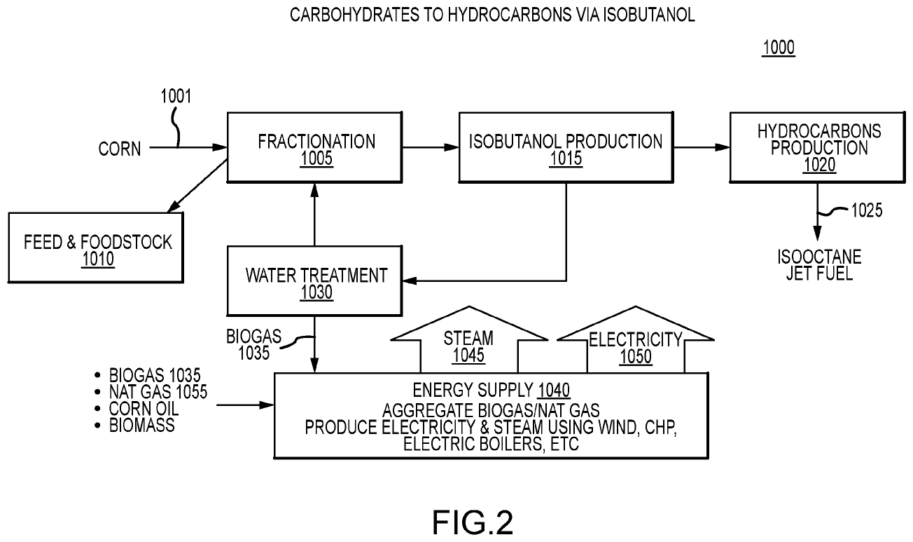

Referring to FIG. 2, a flow chart illustrating a process for converting carbohydrates to hydrocarbons via isobutanol 1000 is shown. A carbohydrate, such as corn 1001, is subjected to fractionation 1005. Fractionation generally refers to the conversion of the carbohydrate into constitute components. The fractionation 1005 products are then used for either feed and footstock 1010, for example although other alternative uses 1010 may be available for different biomasses, or used in isobutanol production 1015. Although described as isobutanol, isobutanol is an example of one possible alcohol that may be used in the overall process. The isobutanol production 1015 products can be used for hydrocarbons productions 1020 or water treatment 1030. The isobutanol production 1015 products used for hydrocarbons production 1020 become isooctane jet fuel 1025. The isobutanol production 1015 product used for water treatment 1030 becomes, in part, a biogas 1035. The biogas 1035 and/or natural gas 1055 may be used to create an energy supply 1040. Optionally, corn oil and/or other biomass can also be used as part of the energy supply 1040. In certain embodiments, any corn oil byproducts may be sold or otherwise disposed. The energy supply 1040 includes aggregate biogas and natural gas as well as, optionally, corn oil and biomass. The energy 1040, in certain embodiments produces electricity 1050 and steam 1045, which, optionally, uses wind power, CHP, electric boilers, combinations thereof, and the like.

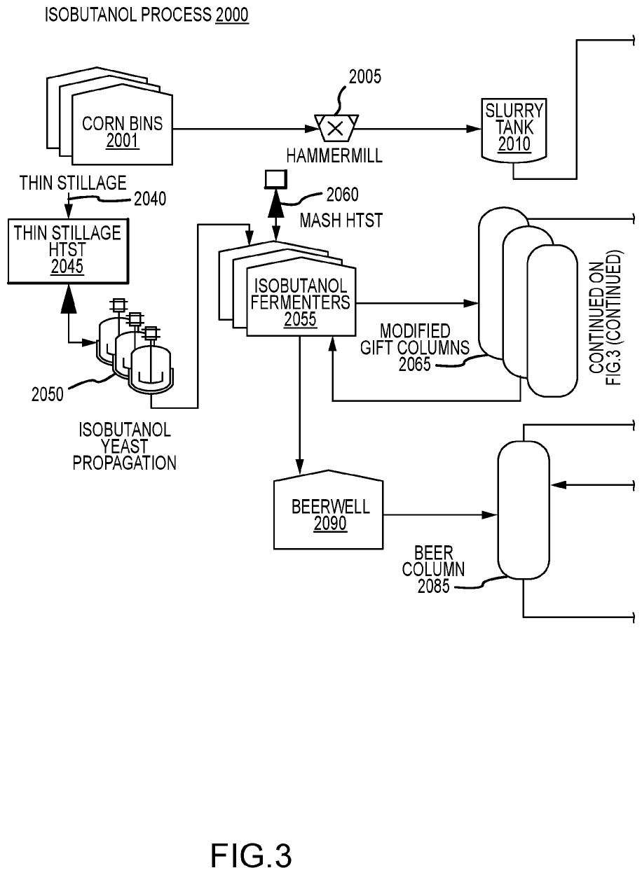

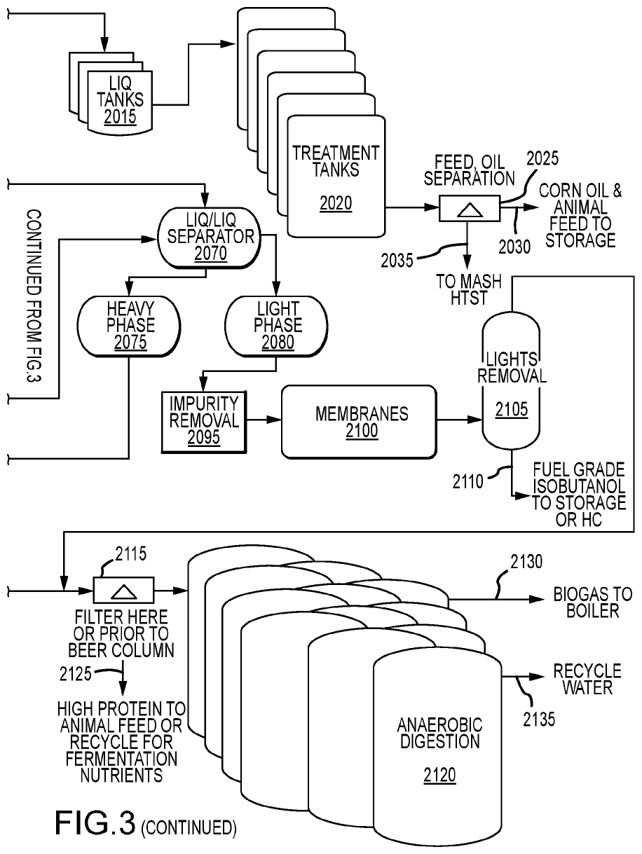

FIG. 3 shows an isobutanol process and system 2000. At the beginning of the isobutanol process and system 2000, a storage vessel 2001, e.g. corn bins, contains a carbohydrate, e.g. corn or high sugar corn, which may be sent to a pulverizer such as a hammer mill 2005, or alternatively carbohydrate thin stillage 2040 may be subjected to thin stillage high-temperature-short-time (HTST) processing 2045. Referring to the carbohydrate that is sent to the pulverizer, it next goes to a slurry tank 2010, followed by liquid tanks 2015 and subsequently treatment tanks 2020. The product of the treatment tanks 2020 undergoes separation such as feed and oil separation 2025, to produce end products such as corn oil and animal feed 2030 for storage, and waste products that are recycled into the process such as via Mash HTST 2035.

Referring to the thin stillage 2040, it is subjected to thin stillage HTST 2045 before use in isobutanol yeast propagation 2050. The product of the isobutanol yeast propagation 2050 then goes to a fermentation unit such as isobutanol fermenter 2055, along with products of Mash HTST 2060. The products of isobutanol fermenters 2055 are sent to modified GIFT columns 2065 and a portion is recycled back to the isobutanol fermenters 2055 or sent to a beer well 2090. What is not recycled from the modified GIFT columns 2065 back into the isobutanol fermenters 2055/beer well 290 is sent to a liquid/liquid separator 2070. Products of the beer well 2090 are sent to a first distillation column 2085, such as beer column 2085. A product of the distillation column 2085 is fed to the liquid/liquid separator 2070, and a product of the distillation column 2085 is fed to a filter unit 2115. The liquid/liquid separator 2070 produces heavy phase 2075 and light phase 2080 liquids. The heavy phase 2075 liquid is sent back to the beer column 2085, such as for a reflux operation. The light phase 2080 liquid is sent to impurity removal 2095, through membranes 2100 for filtration and the like, and into a lights removal unit 2105. The lights removal unit 2105 produces fuel grade isobutanol 2110 for storage or hydrocarbon processing. The product from beer column 2085 fed to filter unit 2115 is combined with lights removal unit 2105 product and filtered either at filter unit 2115, optionally a filter prior to the beer column 2085 may be provided. The output of the filter 2115 is sent to an anaerobic digester 2120. High protein products 2125 from filter 2115 can be used for animal feed, recycled as fermentation nutrients, or otherwise disposed. The anaerobic digester 2120 produces biogas 2130 which can be sent to the boiler of another subsystem, and recycle water 2135, which is recycled into other subsystems.

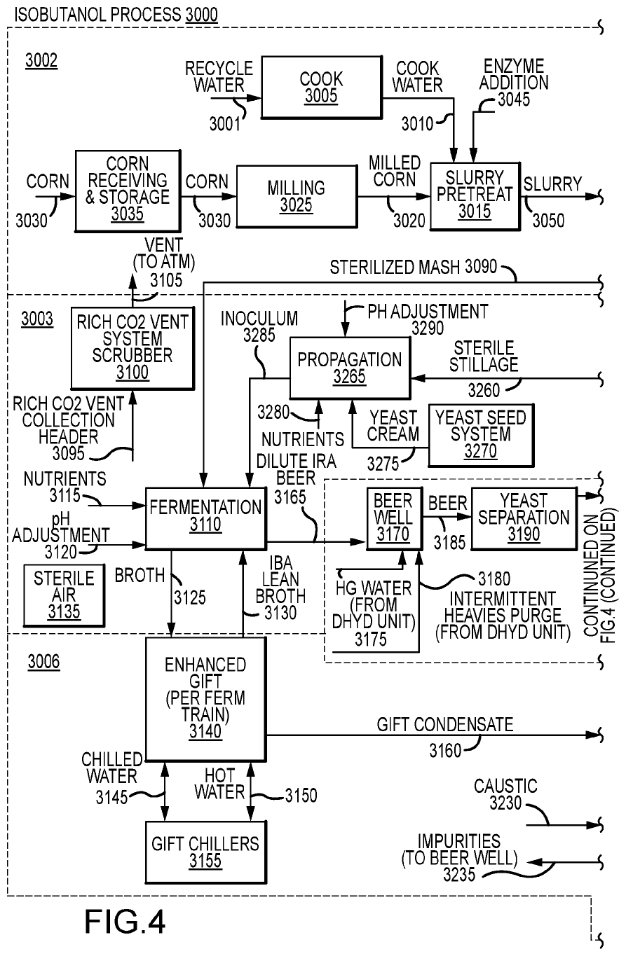

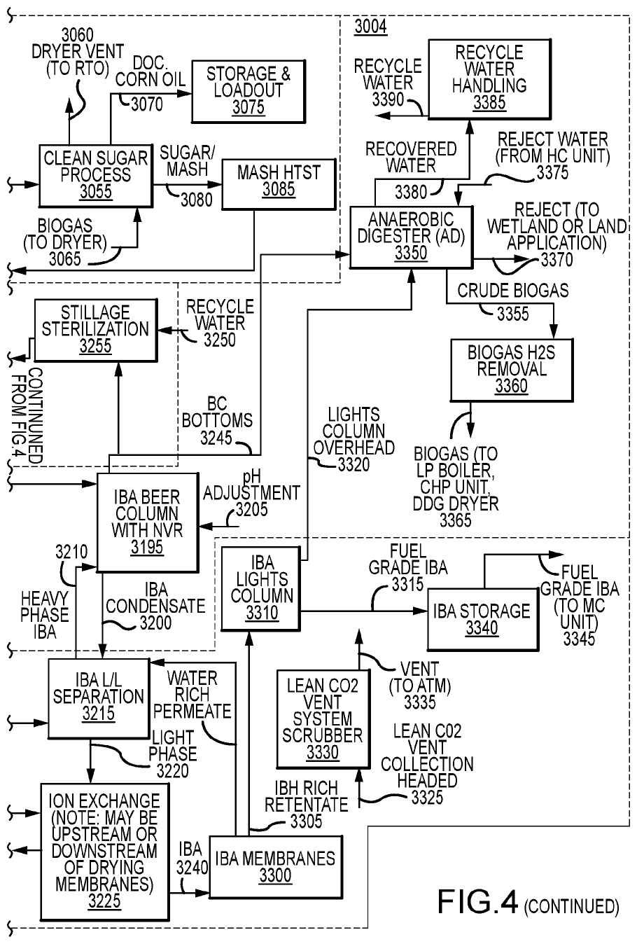

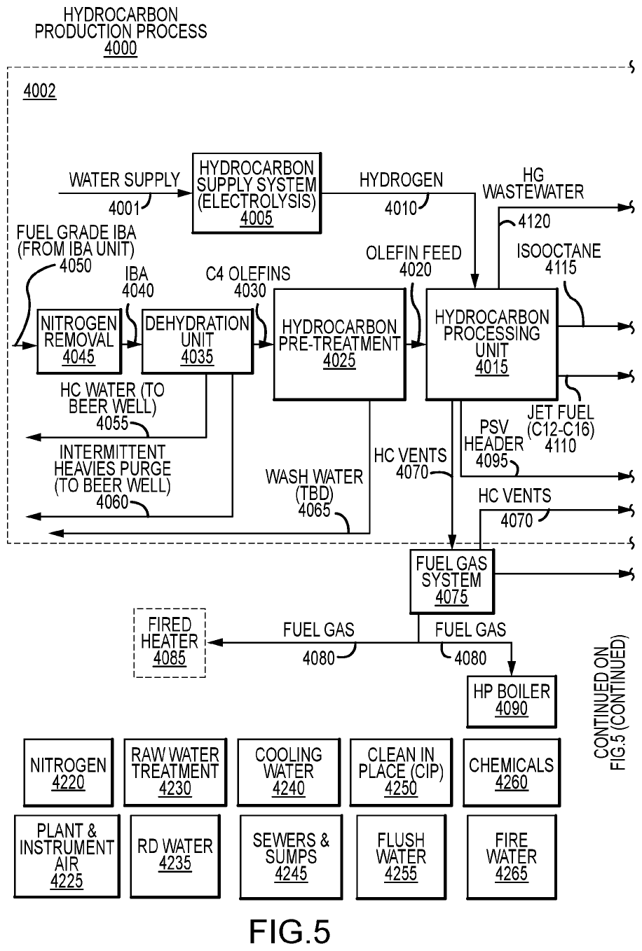

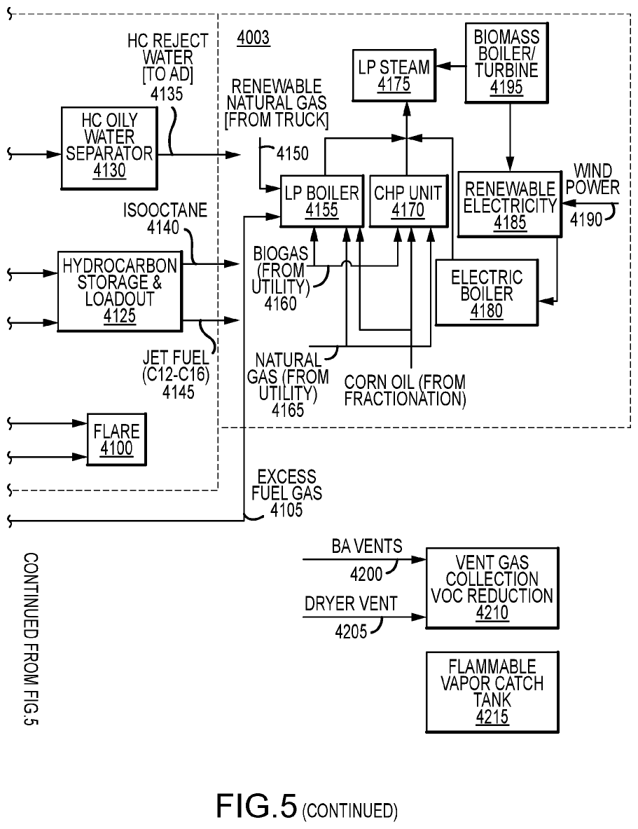

FIG. 4 is a flowchart illustrating an isobutanol production process and system 3000 consistent with a net zero production plant consistent with the technology of the present application that produces, in certain embodiments, transportation fuels. The isobutanol production process and system 3000 flowcharts includes systems and subsystems that having particular structures that, taken together describes a NZ plant consistent with the technology of the represent application. While certain structures, processes, and features of the NZ plant are described with relation to one or more of the identified subsystems, the specific structures, processes, and features may be spread across or contain in other subsystems of the NZ plant without departing from the scope of the present technology. The isobutanol production process and system 3000 includes a fractionation subsystem 3002, a fermentation subsystem 3003, a water treatment subsystem 3004, and an alcohol enrichment system 3006. Products from these subsystems, along with the subsystems shown in FIG. 5, are combined to create the carbon neutral, zero net energy overall production plant that uses the described processes and equipment.

In the fractionation subsystem 3002, recycle water 3001 is put through cook 3005 to create cook water 3010. Additionally, a carbohydrate such as corn 3030 moves through a receiving and storage system 3035, and subsequently milling 3025 to make milled corn 3020. The milled corn 3020 and cook water 3010, along with added enzyme 3045, are placed into slurry pretreatment 3015. The resultant slurry 3050, along with biogas 3035, goes into clean sugar process 3055. The clean sugar process 3055 has a dryer vent to atmosphere 3060, and produces products 3070, such as dried distiller's grain (DDG) and corn oil for storage and load out 3075, and sugar/mash 3080 for Mash HTST 3085 processor, which treats the sugar/mash 3080. The sterilized mash 3090 from the Mash HTST 3085 is then put into the fermentation subsystem 3003.

In the fermentation subsystem 3003, there is sterile air 3135, and additionally, air from a rich CO₂ vent collection header 3095. The rich CO₂ air is scrubbed by a rich CO₂ vent system scrubber 3100 and the resultant air is vented to the atmosphere 3105.

The sterilized mash 3090, received from fractionation subsystem 3002, along with nutrients 3115 and a pH adjustment agent 3120 are placed into fermentation 3110 structure. Additionally, recycle water 3250 is directed into stillage sterilization 3255, and the resulting sterile stillage 3260, along with nutrients 3280, a pH adjustment agent 3290, and a microorganism 3275, such, for example, as yeast cream 3275, from a microorganism propagation unit 3270, such as, for example, yeast seed system 3270, are placed into propagation 3265. The propagation 3265 produces an inoculum 3285. The inoculum 3285 is fed to fermentation 3110, along with the above constituent parts. The fermentation 3110 unit produces at least a first portion and a second portion. The fermentation 3110 unit second portion includes at least a fermentation broth 3125 and. The fermentation 3110 unit first portion includes at least a dilute beer 3165 (or an alcohol 3165), such as isobutanol beer 3265. The fermentation broth 3125 is provided to the modified/enhanced GIFT 3140 of the alcohol enrichment subsystem 3006, described below.

A portion of the dilute beer 3165, such as for example, a dilute isobutanol beer 3165, from fermentation 3110 may be used for water recovery in the water treatment subsystem 3004, described below. A portion of the dilute beer 316 may be used by the alcohol enrichment system 3006, as described below. Prior to water recover or alcohol enrichment, however, beer well 3170 receives the dilute beer 3165. HC water from a DHYD unit 3175, and intermittent heavy component purge from the DHYD unit 3180, are fed to beer well 3170 to create beer 3185 (or undiluted beer 3185). The beer 3185 moves through yeast separation 3190 unit a beer column with MVR 3195, such as, for example, an isobutanol (IBA) beer column with MVR 3195. The the beer column with MVR 3195 receives a pH adjustment agent 3205. The resulting beer column with MVR 3195 condensate goes to the alcohol enrichment subsystem 3006 via a light/light separation unit 3215, such as, for example, an IBA light/light separation unit 3215. The heavy phase 3210 from the light/light separation unit 3210 is recycled back into the beer column with MVR 3195. The beer column with MVR 3195 produces BC bottoms 3245, which is used in the stillage sterilization 3255. A portion of the BC bottoms 3245 is used in the water treatment subsystem 3004, explained below.

The alcohol enrichment system 3004 receives the fermentation broth 3125 from the fermentation subsystem 3003. An enhanced GIFT 3140 receives the fermentation broth 3125. The enhanced GIFT passes chilled water 3145 and hot water 3150 back and forth between GIFT chillers 3155. The enhanced GIFT 3140 produces lean broth 313, such as, IBA-lean broth 3130, that is provided to the fermentation 3110 of the fermentation subsystem described above and a GIFT condensate 3160. A separation unit 3125, such as IBA light/light separation unit 3215, receives the GIFT condensate 3160 to produce, among other things, a light phase IBA 3220 that goes to ion exchange 3225, as well as the heavy phase 3210 received by the beer column with MVR 3195 described above. The ion exchange 3225 may be upstream or downstream of drying membranes 3300, such as IBA membranes 3300. Caustic agents 3230 are added to the ion exchange 3225, and resulting impurities 3235, optionally, are recycled to the beer well 3170. IBA 3240 from the ion exchange 3225 also moves through IBA membranes 3300 to create water-rich permeate 3306 that is recycled to IBA light/light separation 3215 and IBA-rich retentate 3305 that is sent to an IBA light column 3310. The IBA lights column 3310 creates fuel grade IBA 3315, which can be sent to IBA storage 3340 before being sent to the HC unit as fuel grade IBA 3345. Lights column overhead 3320 from the IBA lights column 3310 is fed to the anaerobic digester (AD) 3350 of the water treatment subsystem 3004, described below. The alcohol enrichment subsystem 3006 may include a lean CO₂ vent collection header 3325 that is vented to atmosphere 3335 through a lean CO₂ vent system scrubber 3330.

The water treatment subsystem 3004, as described above, receives BC bottom 3245 from the fermentation subsystem 3003 and lights column overhead 3320 from the alcohol enrichment subsystem 3006 at AD 3350. The AD 3350 also uses reject water from the HC unit 3375, and creates crude biogas 3355, recovered water 3380, and reject waste products 3370. The rejected waste products are disposed, such as, for example, by being sent to wetlands or land applications. The crude biogas 3355 is sent to biogas H₂S removal 3360 unit, to create biogas 3365 that may be sent to the LP broiler, CHP unit, DDG dryer, a combination thereof, or the like. The recovered water 3380 is sent to recycled water handling 3385 to create recycle water 3390.

Shown in FIG. 5 is a hydrocarbon production process and system 4000. The hydrocarbon production process and 4000 includes a hydrocarbon production subsystem 4002 and an energy management subsystem 4003. The hydrocarbon production process and system 4000 also provides certain ancillary component parts and processes that may or may not be specifically incorporated into the hydrocarbon production subsystem 4002 and energy management subsystem 4003. The hydrocarbon production process and system 4000 is coupled with the isobutanol process and system 3000 to form part of a carbon neutral, net zero energy overall process and plant. The hydrocarbon production subsystem 4002 intakes a water supply 4001, which includes recycled water from other subsystems such as recycle water 3390, described above, into a hydrogen supply system 4005 to form hydrogen 4010. Additionally, fuel grade IBA 4050, which includes IBA from another subsystem, such as fuel grade IBA 3345, described above, undergoes nitrogen removal 4045 to produce IBA 4040. A dehydration unit 4035 receives the IBA 4040 and produces hydrocarbon water 4055 and intermittent heavies purge 4060 to be sent to the beer well, such as beer well 3170 as described above. The dehydration unit 4035 also produces a feed 4030, such as, for example C₄ Olefins 4030, to hydrocarbon pre-treatment 4025. The hydrocarbon pre-treatment derives olefin feed 4020 and wash water 4065, which may be, optionally, used in various systems described herein. The olefin feed 4020, along with hydrogen 4010, described above, are sent to a hydrocarbon processing unit 4015. The hydrocarbon processing unit 4015 produces hydrocarbon wastewater 4120, which is sent to a hydrocarbon oily water separator 4130 to create hydrocarbon reject water 4135 to be sent to the AD 3350 described above. The hydrocarbon processing unit 4015 also produces hydrocarbon vent products 4070 that leave the hydrocarbon production subsystem 4002 via a fuel gas system 4075 to produce fuel gas 4080, described below, and the vent products may be provided to a PSV header 4095 and subsequently received in a flare 4100 system. Additionally, the hydrocarbon processing unit 4015 produces isooctane 4115 and C₁₂-C₁₆ jet fuel 4110, both of which are sent to hydrocarbon storage and loadout 4125. Isooctane 4140 and C₁₂-C₁₆ jet fuel 4145 can then be obtained from hydrocarbon storage and loadout 4125.

The fuel gas system 4075, described above, also provides fuel gas 4080 to a fired heater 4085 and a HP boiler 4090, where the fuel gas 400 is used.

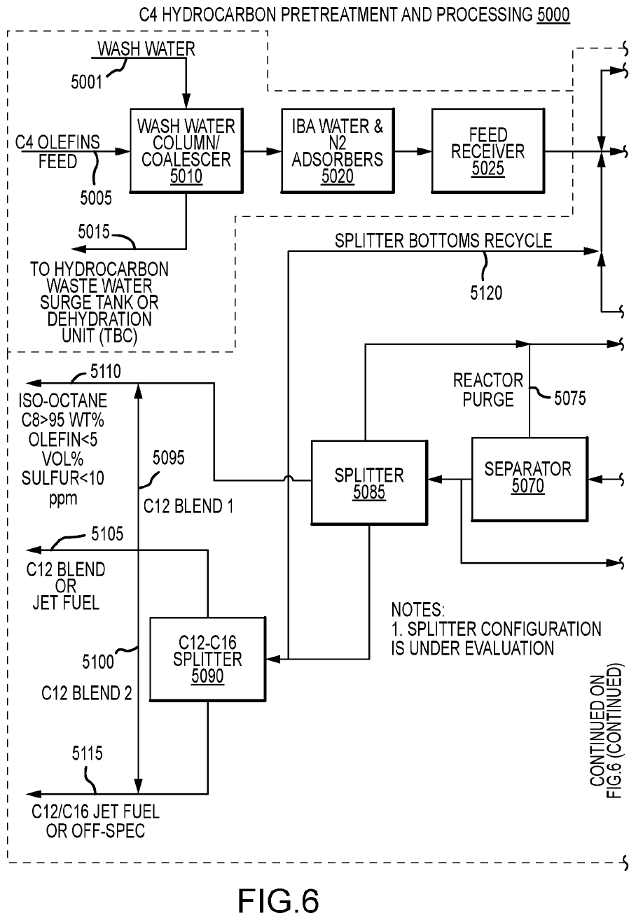

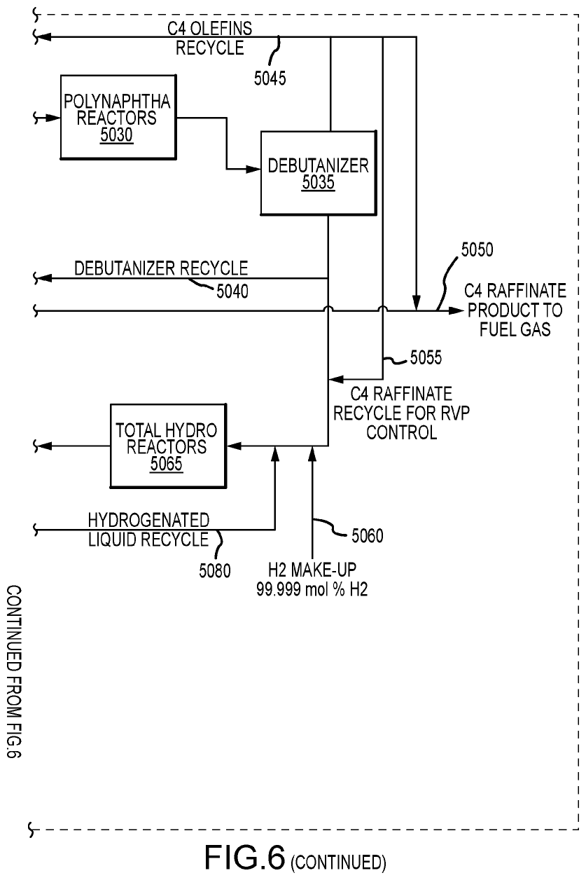

Excess fuel gas 4105 from fuel system 4075, to the extent there is excess fuel gas, may be provided to the energy management subsystem 4003 via an LP boiler 4155. The LP boiler 4155 also intakes, as needed, renewable natural gas 4150, biogas 4160 from the AD 3355, described above, and natural gas 4165 from a utility system, again as needed. A CHP unit 4170 intakes biogas 4160, natural gas 4165, as needed, and, optionally, corn oil from fractionation. The output of the LP boiler 415 and the CHP unit 4170, create LP steam 4175. LP Steam 4170, which may be a steam header, may receive input from a biomass boiler/turbine 4195 and an electric boiler 4180, which electric boiler 4180 may use renewable energy sources to power the electric boiler 4180. The renewable energy sources include wind power 4190, photovoltaic panels (not shown) or energy created by the turbine of the biomass boiler/turbine 4195. The hydrocarbon production process and system 4000 also includes some ancillary systems and process. For example, the hydrocarbon production process and system 400 includes, among other things, a vent gas collection and volatile organic compound (VOC) reduction unit 4210 which intakes from IBA vents 4200 and dryer vent 4205. Other components of the hydrocarbon production process and system 4000 include a flammable vapor catch tank 4215, nitrogen 4220, plant and instrument air 4225, raw water treatment 4230, reverse osmosis (RO) water 4235, cooling water 4240, sewers and pumps 4245, a clean in place (CIP) unit 4250, flush water 4255, chemicals 4260, and firewater 4265. The hydrocarbon pre-treatment 4025 and hydrocarbon processing unit 4015 are described in further detail in FIG. 6.

FIG. 6 illustrates more detail of the hydrocarbon pre-treatment 4025 and hydrocarbon processing unit 4015 as a hydrocarbon pretreatment and processing scheme 5000, which describes features, processes, and structure. Initially, a wash water column/coalescer 5010 receives wash water 5001 and a C₄ olefin feed 5005. The wash water column/coalescer 5010 feeds to a hydrocarbon waste water surge tank or dehydration unit (TBC) 5015 and to IBA water and nitrogen adsorbers 5020. The IBA water and nitrogen adsorbers 5020 feeds a feed receiver 5025 prior to combining with a C₄ olefins recycle 5045, a debutanizer recycle 5040, and a splitter bottoms recycle 5120. Polynaptha reactors 5030 received the aforementioned combination and feeds product to a debutanizer 5035. Products of the debutanizer 5035 either enter the debutanizer recycle 5040 or combine with C₄ raffinate recycle for RVP control 5055, hydrogen made up of 99.999 mol % H₂ 5060, and hydrogenated liquid recycle 5080 to enter total hydro reactors 5065.

The total hydro reactors 5065 feed a separator 5070 that produces a reactor purge 5075 and the hydrogenated liquid recycle 5080, some of which is provided to the total hydro reactors 5065 (above) and some of which is provided to splitter 5085. The reactor purge 5075, an output of the splitter 5085, and C₄ olefins recycle 5045 combine to produce C₄ raffinate product to fuel gas 5050 The output of separator 5070 not used as hydrogenated liquid recycle 5080 is received by splitter 5085 that creates a splitter bottoms recycle 5120, which feeds back into the debutanizer recycle 5040 and feeds to C₁₂-C₁₆ splitter 5090, and isooctane 5110. The C₁₂-C₁₆ splitter 5090 creates a C₁₂ blend or jet fuel 5105 or C₁₂/C₁₆ jet fuel 5115. Furthermore, a first C₁₂ Blend 5095 can be created from isooctane 5110 and C₁₂ blend or jet fuel 5105, and a second C₁₂ blend 5100 can be created from C₁₂ blend or jet fuel 5105 and C₁₂/C₁₆ jet fuel 5115.

### Process Scheme for a Energy Efficient Hydrocarbon Production Process Using Ethanol as an Alcohol Intermediate

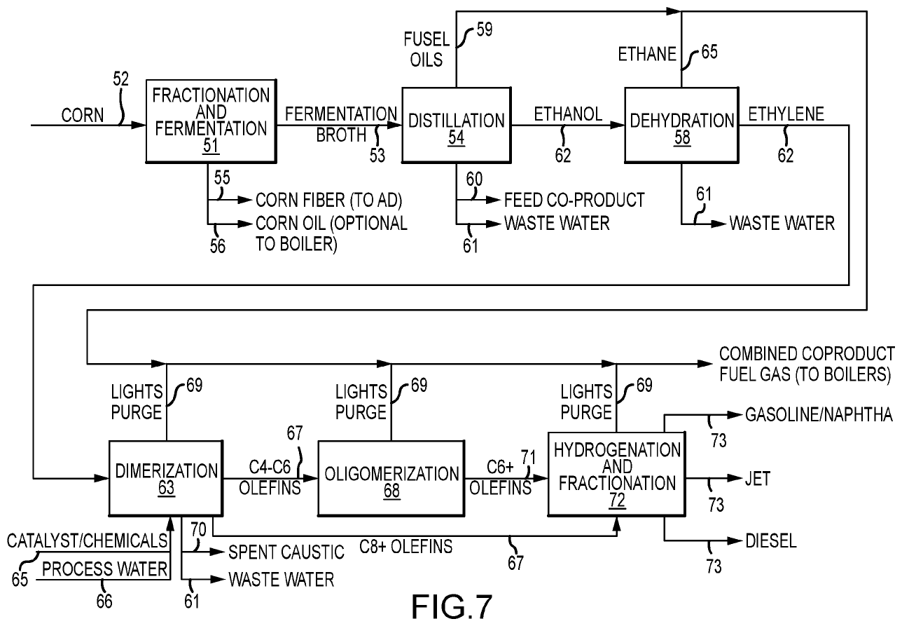

A schematic of a low energy production process well-tailored to Jet and Diesel fuels derived from an ethanol intermediate is shown in FIG. 7. The feedstock should be selected such that the input feedstock has zero or a negative carbon footprint.

FIG. 7 is a process and equipment diagram 50. The process and equipment diagram 50 shows a fractionation and fermentation unit 51 receives a carbohydrate 52, such as corn 52. The fractionation and fermentation unit 51 outputs a fermentation broth 53 that a distillation unit 54 receives. The fractionation and fermentation unit 51 also outputs corn fiber 55 and corn oil 56. The corn fiber 55 may be provided to an anaerobic digester (explained elsewhere herein). The corn oil 56 may be used as fuel or otherwise disposed. The distillation unit 54 outputs an alcohol 57, such as ethanol 57, that is received by dehydration unit 58. The distillation unit 54 outputs fusel oils 59 that are combined with other combustibles and sent to a boiler or the like. The distillation unit 54 also outputs a feed co-product 60 and wastewater 61. The dehydration unit 58 dehydrates the alcohol 57 and outputs ethylene 62 that a dimerization unit 63 receives. The dehydration unit 58 also outputs ethane 64 that is combined with the fusel oils 59 and other combustibles and additional wastewater 61. The dimerization unit 63 receives the ethylene 62, catalysts and/or chemicals 65 to facilitate operation of dimerization unit 63, and water 66 to produce C₄-C₆ olefins 67 and C₈₊ olefins 67. The C₄-C₆ olefins 67 are received by a oligomerization unit 68. The dimerization unit 63 outputs lights purge 69 that are combined with the other combustibles. The dimerization unit 63 also outputs byproducts that include spent caustic agents 70 and additional wastewater 61. The oligomerization unit 68 receives the C₄-C₆ olefins 67 to produce C₆₊ olefins 71 received by a hydrogenation and fractionation unit 72. The oligomerization unit 68 also products additional lights purge 69 that are combined with other combustibles. The hydrogenation and fractionation unit 72 receives the C₄-C₆ olefins 67 and C₈₊ olefins 67 to produce transportation fuels 73. The hydrogenation and fractionation unit 72 also outputs lights purge 69 that are combined with other combustibles.

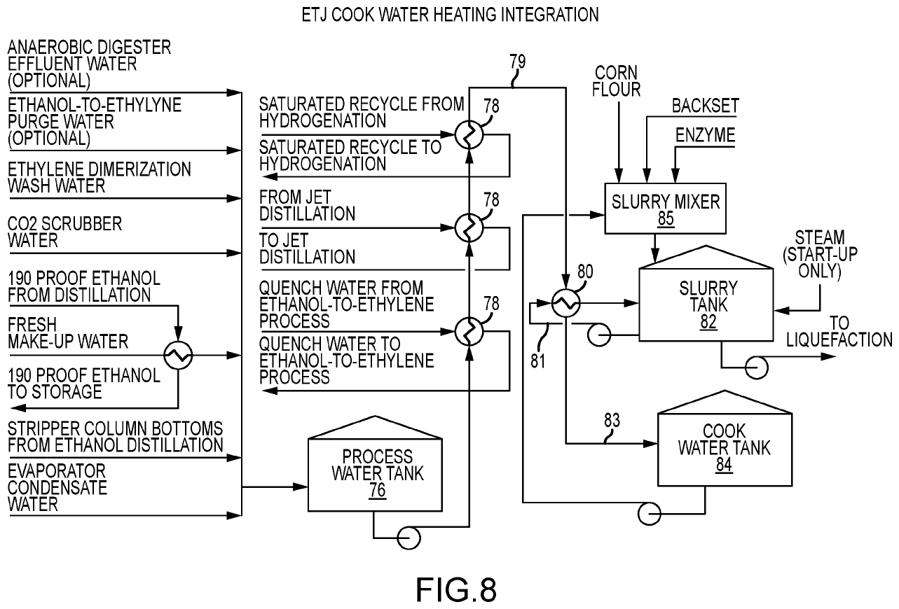

As disclosed herein, low carbon intensity (CI) corn or some other low CI carbohydrate, carbohydrate-rich feedstock is fed into the process as shown in FIG. 7. In the case of corn, for example, the feedstock is simply milled into a flour of desired particle size before being fed forward. The flour is mixed with warm water and enzymes (for example, alpha amylase and/or other suitable enzymes) before being fed into a liquefaction system that provides enough residence time for the starch in the corn flour to liquefy into polysaccharides. In some aspects, enzymes, such as cellulolytic enzymes, cellulase, protease, pectinase, xylanase, α-1-arabinofuranosidase, and/or phytase can be used. In conventional corn-based ethanol dry mill facilities, heating the corn flour/water mixture before liquefaction is a utility-intensive process in that large amounts of steam are directly injected into the slurry tank to achieve a target temperature. To help minimize steam and freshwater usage, warm water from the ethanol distillation system, warm water from the evaporator system, and warm thin stillage (referred to as “backset”) are recycled to the slurry tank from downstream. Additional water is oten needed though to achieve the target percent total solids in the slurry tank. CO₂ scrubber bottoms is a common cold-water stream that is fed into the slurry tank. If anaerobic digestion is being used for the project, the treated AD effluent water can also be fed to the slurry tank. In some cases, fresh make-up water such as city water could also be fed into the slurry tank. At their sources, these water streams are relatively cold and will increase the load of steam needed in the slurry tank. For this low energy process to produce biofuels, design features are being included to use excess heat from other parts of the NZ plant to heat the cold-water streams feeding the slurry tank thereby decreasing steam usage to the point of only being needed during start-up. FIG. 8 shows an example of the heat integration scheme being used to pre-heat the water feeding the slurry tank. Other configurations of FIG. 8 to integrate heat generated from one part of the NZ plant to another are possible and within the scope of the present application.

FIG. 8 shows a process and equipment diagram 75 for cook water heating integration. The diagram 75 shows one possible equipment and process configuration and others are possible and within the spirit and scope of the present technology. Liquid, heated by other processes associated with the net zero carbon footprint plant to produce transportation fuels and described elsewhere herein, are received by a process water tank 76. The process water tank 76 contains heated water 77 or process water 77 for re-use by the plant, which conserves the heat energy. The process water 77 may be used direction from the process water tank 76 or it can be further heated, and pressurized, by one or more heat exchangers 78, described further below as exemplary exchangers, where the heat exchangers 78 heat the process water 77 to superheated process water 79. To be safely stored, a slurry heat exchanger 80 uses the heat from superheated process water 79 to heat the slurry 81 in slurry tank 82. The slurry heat exchanger 80 cools and depressurizes the superheated process water 70 to cook water 81 that is stored in cook water tank 81. A slurry mixer 82 uses the cook water to combine cook water 81, corn flour (or the like), backset (described elsewhere herein) and enzymes to produce the slurry 81.

The anaerobic digester, such as AD 3350 is optional. As the AD is optimal, it may not be available to produce an effluent water stream as shown. A ethanol reactor creates an ethanol-to-ethylene purge water stream, which reactor is optional. The ethanol reactor converts ethanol to ethylene and water. This stream can be used to offset the freshwater fed to the CO₂ scrubber or it can be fed directly into the process water tank as shown in FIG. 8. An ethylene dimerization wash water is generated from washing the butenes formed from dimerizing ethylene in preparation for sending the butenes to the oligomerization process that produces C₈, C₁₂, and C₁₆ olefins. The ethanol-to-ethylene process receives 190 proof ethanol as a feed and, for safety reasons, it is cooled prior to going into intermediate storage by a water chiller in this example, other heat exchangers could be used. The hot 190 proof ethanol is cross-exchanged with cold fresh make-up water to that is heated by the hot 190 proof ethanol, which conserves the heat drawn from the hot 190 proof ethanol. The heated water is provided to the process water tank. The stripper column bottoms is a relatively clean water stream that is generated in the production of 190 proof ethanol. It is warm when it comes from ethanol distillation and is fed directly into the process water tank. The evaporator condensate water is generated in the process of removing water from thin stillage to produce syrup. The syrup is ultimately blended with wet cake and either sold as-is for animal feed or, optionally, fed to a dryer to produce DDGS (Dry Distillers' Grains with Solubles). The evaporator condensate water is warm and contains low levels of impurities such as organic acids. It is fed directly to the process water tank.

The combined water streams in the process water tank are warm, but additional heat is needed to achieve the target temperature in the slurry tank. A broad set of cooling services in the ethanol-to-jet process were evaluated where the heat removed by a cooling water cooled exchanger or air-cooled exchanger would instead be used to heat the water leaving the process water tank. Cooling services were evaluated based on cooling load, temperature, operability, and safety. Although there were other services with higher cooling loads or useful temperature ranges, the three services that satisfied all criteria including operability and safety are shown in FIG. 8. Cooling services related to the exothermic heat of reaction generated from dimerizing ethylene and oligomerizing butenes were evaluated but were ultimately eliminated as heat sources for cook water due to being too cold or presenting safety/operability concerns in managing reaction temperatures. The three cooling services shown in FIG. 8 are arranged in a way that allows for the process water to be heated from about 67° C. to about 112° C. in three steps. The process water leaving the cross-exchanger with saturated recycle from hydrogenation is above the boiling point of water at atmospheric pressure. The process water will be back-pressure controlled at this point making it superheated. It is necessary to accumulate the hot process water in a surge tank, as shown by the cook water tank in FIG. 8, to manage process fluctuations, but the water cannot be stored superheated safely or economically and a portion will vaporize if it is stored at atmospheric pressure. For this reason, the superheated process water streamed is cross-exchanged with a circulation loop from the slurry tank to decrease the temperature of the process water to below the atmospheric boiling point before being fed to the cook water tank which is at atmospheric pressure. The cook water tank, as shown in FIG. 8, operates at approximately 95° C. The cook water is fed to the slurry mixer where it is combined with corn flour, backset, and enzymes. Once combined, the slurry mixer injects the combination to the slurry tank. In total the heat integration scheme, as shown in FIG. 8, saves at least 33 MMBTU/hr of steam for a 100 million gallon per year ethanol capacity (anhydrous basis, 8000 annual operating hours), which equates to 39 MMBTU/hr of natural gas assuming a boiler efficiency of 85%. As mentioned above, other configuration to use heated water from other parts of the NZ plant to store heated process water and to further heat the process water are possible and within the spirit and scope of the present application.

Optionally, the stream leaving liquefaction can undergo additional processing to reduce the particle size of corn particles before being fed downstream. This is advantageous because it helps liberate additional corn oil form the corn particles making more corn oil available for recovery downstream, although recovering energy via the corn oil is optional as the corn oil is usable in other carbon reducing manners. In the case of this low energy process for producing biofuels, the corn oil will be recovered and used as a fuel for the boiler system which helps reduce the carbon intensity of the final jet product as well as the naphtha and diesel coproducts.

The stream leaving liquefaction feeds into the fermenter tanks where it combines with additional enzymes and yeast. The fermentation is carried out in a conventional way. The liquified starch is simultaneously saccharified to form monosaccharides which are then consumed by the yeast to form ethanol and CO₂ gas. The CO₂ gas is vented from the fermenters to a scrubber system that uses water to scrub any ethanol or other water-soluble impurities that have been carried with the CO₂ vapor. The fermentations are carried out in batches. When a fermentation batch is complete the monosaccharides are nearly gone with a small number of residual sugars left behind. The ethanol concentration is about 13.0 wt %, 13.5 wt %, 14.0 wt %, 14.5 wt %, 15.0 wt %, or 15.5 wt %. In general the yeast does not consume the corn fiber, corn oil, corn protein, ash, and other corn-based components that are in the fermenter broth with the exception of a small amount of nutrients consumed as the yeast continue to multiply during fermentation. The ethanol, water, yeast, and corn-based components are transferred to a large surge tank referred to as the beer well. A stream of beer is continuously fed out of the beer well downstream into ethanol distillation where the ethanol is recovered while most of the water, corn components, yeast, and enzymes are sent downstream in what is called whole stillage.

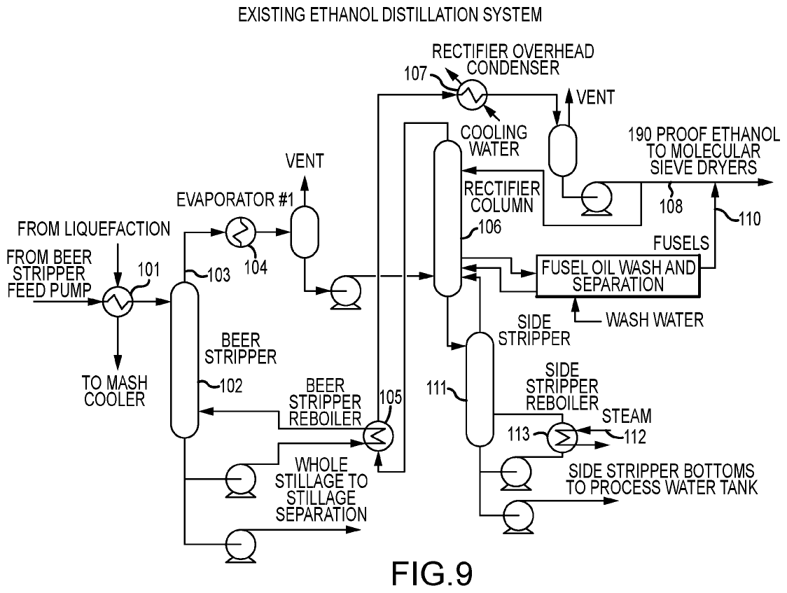

Ethanol distillation can be carried out in several ways. At corn ethanol dry mill facilities that produce fuel-grade ethanol, the ethanol distillation system is almost always heat-integrated with the evaporator system in some way to reduce the amount of fresh steam needed to run the process. FIG. 9 shows one example of a commercially available technology for distilling fuel-grade ethanol.

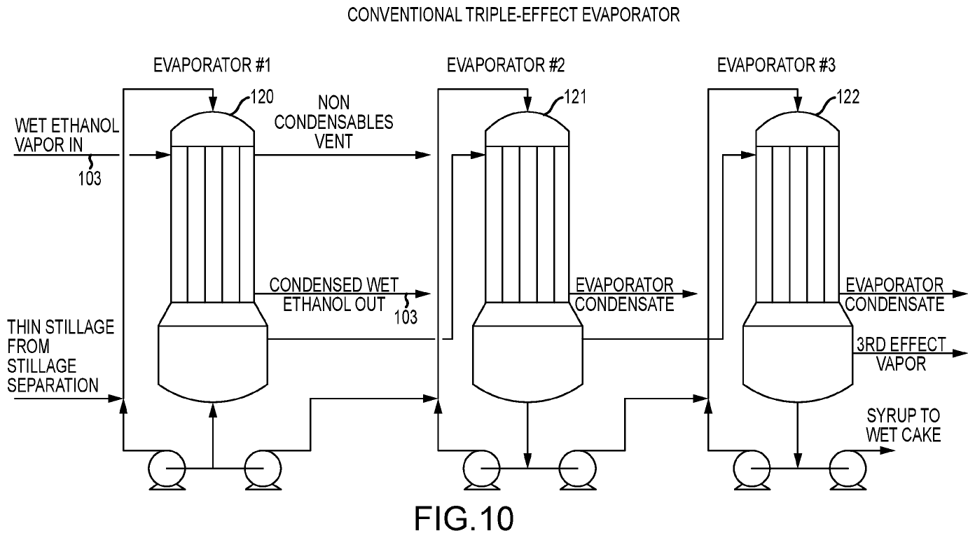

Like all available technologies, the one in FIG. 9 has features to facilitate operation. The beer from the beer well is almost always cross-exchanged with the stream leaving liquefaction to pre-heat the beer on its way to distillation and to save on cooling water demand when cooling the liquified mash before fermentation. FIG. 9 shows the wet ethanol vapor removed at the top of the beer stripper being condensed in the first evaporator of a multi-effect evaporator system, shown in FIG. 10. The heat removed from the wet ethanol vapor is used to drive water vapor away from the thin stillage in the first evaporator. The water removed in the first evaporator is used to drive the separation in the second-effect evaporator and so on. Evaporation is an energy intensive process, but the scheme shown in FIGS. 9 and 10 is such that the evaporator system requires no fresh steam during operation. FIG. 10 shows a typical triple-effect evaporator configuration that can be used.

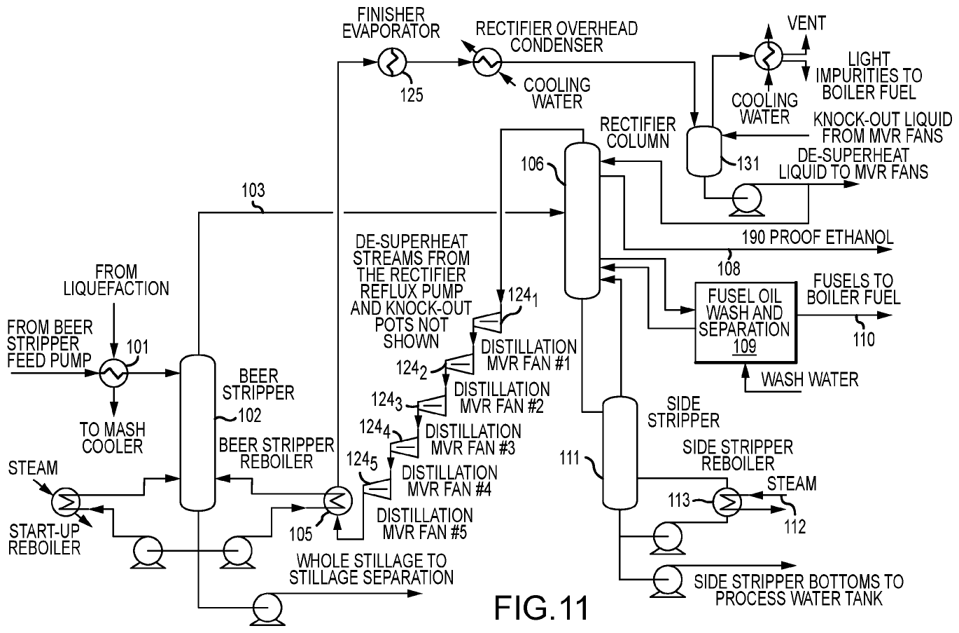

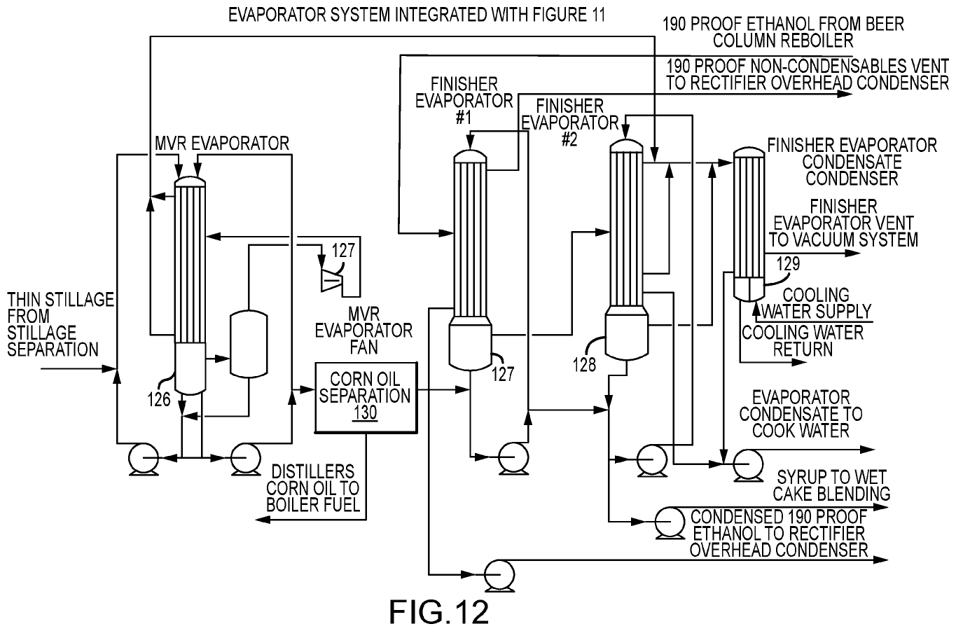

FIG. 11 shows an energy conservation improvement to the distillation system shown in FIG. 9 consistent with the technology of the present application. In particular, it has been found that condensing the overhead vapor from the beer stripper allows the pressure and temperature of the overhead vapor/liquid to be heated using one or more pump. The pressure can be such that the condensed overhead vapor of the rectifier operates at a higher pressure and temperature such that the 190 proof ethanol can run the beer column reboiler. The beer column reboiler has a substantial duty, so this heat integration saves on fresh steam and reduces the overall external energy required by the NZ plant. The issue with condensing the vapor at the top of the beer column before feeding into the rectifier column is that most of the feed into the rectifier now needs to be re-vaporized to drive the ethanol to the top of the rectifier. The energy for re-vaporization comes from a large amount of fresh steam used for the side stripper reboiler.

For this lower energy process to produce biofuels, it is desirable to reduce all fresh steam and natural gas usage as much as possible, so even a scheme like the one in FIGS. 9 and 10 have opportunities for energy reductions. For this lower energy process to produce biofuels, a scheme like the one shown in FIG. 11 may be deployed. FIG. 11 shows a possible configuration although other configurations are within the spirit and scope of the present application.

FIG. 11 shows the distillation system with MVR fans, see beer column with MVR fans 3195 in FIG. 4 above. The MVR fans shown in FIG. 11 may be considered a mechanical vapor recompression (MVR) system. FIG. 11 identifies features that have been included to remove fermentation byproducts from the 190 proof ethanol stream that would present issues in the ethanol dehydration reaction and purification. The 190 proof ethanol is pulled from a draw tray one or more stages down from the top of the rectifier column (notice, columns as used in the present application in context may be distillation towers). This enables acetaldehyde to accumulate in the rectifier overhead, away from the 190 proof, and be purged out in the lights purge stream shown in the FIG. 11. The acetaldehyde can form unwanted byproducts like CO₂ during the ethanol dehydration reaction, so keeping it out of the 190 proof ethanol is desired, which is why the 190 proof ethanol is drawn from a draw tray condensed below the top of the rectifier column.

As shown in FIG. 11, the fusel stream is not recombined with the 190 proof ethanol stream as it is in a conventional corn dry mill plant that produces fuel-grade ethanol. The fusel oil stream contains high concentrations of C₄ and C₅ alcohols. These C₄ and C₅ alcohols react in the ethanol dehydration reaction to form their respective mono-olefins. These olefins are ultimately removed from the ethylene produced along with other oxygenated byproducts and, optionally, burned as fuel to offset other heating demands of the process. The fusels have more heating value as a fuel before being converted to olefins, so for this low energy process to produce biofuels they are not combined with the 190 proof ethanol. Table 1 shows examples of the fusel oil stream composition and lights purge composition, as shown in FIG. 11.

TABLE 1

| | Lights Purge | Fusels |
|---|---|---|
| Component | wt % | wt % |
| Ethanol | 70.8905 | 25.6346 |
| Water | 3.6641 | 27.6144 |
| Carbon Dioxide | 8.2980 | |
| Acetaldehyde | 14.6326 | 0.0032 |
| Acetal | 0.0950 | 0.0089 |
| Ethyl Acetate | 2.0928 | 0.0012 |
| Methanol | 0.1249 | 0.0010 |
| N-propanol | | 0.0897 |
| N-butanol | | 0.9071 |
| 2-butanol | | 0.0696 |
| Isobutanol | | 7.8546 |
| Isoamyl Alcohol | | 22.7177 |
| Amyl Alcohol | | 15.0954 |
| Acetone | 0.1096 | 0.00003 |
| Isopropanol | 0.0001 | 0.0001 |
| 1-pentanol | | 0.0011 |
| Diethyl Ether | 0.0916 | |
| Tert-butanol | 0.0008 | 0.0012 |
| Total | 100.000 | 100.000 |

The lights purge plus fusels combined are able to offset as much as 27 MMBTU/hr (higher heating value) of thermal demand for the project. Burning the corn oil as fuel provides another 62 MMBTU/hr (higher heating value) for producing steam.

The ethanol distillation for this lower energy process to produce biofuels differs from what a conventional corn ethanol dry mill may do in a number of ways. One clear distinction, as shown in FIG. 11, is the addition of a mechanical vapor recompression (MVR) scheme centered around five MVR fans placed in series in between the rectifier overhead vapor and the beer column reboiler. MVR mechanically compresses a vapor which simultaneously increases its temperature to a more useful range and increases the pressure such that the vapor will condense at a higher temperature. This effect makes available the latent heat of condensation of the vapor to drive something like the beer column reboiler as shown in FIG. 11. At least three, four, and five or more MVR fans are shown because both temperature and pressure rise across an MVR fan is in the range of 11° C. and 6.5 psi, respectively. It is believed, a total pressure rise of 20 psi, 25 psi, 30 psi, 35 psi, 40 psi, 45 psi, 50 psi or more is preferred across the fans for the vapor to have a condensing range that is higher temperature than what the bottom of the beer column will be operating. The temperature rise across the fans is controlled by injecting high proof liquid from the rectifier pump into the feed to each fan. In one embodiment, the total number of MVR fan is preferably five. A single MVR fan provides benefits. In general, certain embodiments of the present technology will include at least three (3) MVR fans. Other embodiments of the present technology will include no more than five (5) MVR fans. In still other embodiments of the present technology, three (3) to five (5) MVR fans are provided.

The exemplary system configuration shown in FIG. 11 provides five (5) MVR fans to increase the pressure and temperature of the rectifier overhead vapor to a useful range where it can be used to run the reboiler of the beer stripper.

The five (5) stage MVR, for example, as depicted in FIG. 11, have been found to be the most cost effective to get the required thermal energy savings, for at least the reasons, for example:
i. MVR fans heat/pressurize the rectifier column overhead sufficiently to drive the beer reboiler without the use of external energy,
ii. More than five (5) MVR fans drove the cost up without increasing the conservation of energy,
iii. Less than five (5) MVR fans in the present configuration as shown in FIG. 11 did not provide sufficient increases the drive the beer reboiler without the introduction of external energy,
iv. A compressor costs nearly three times the cost of five (5) MVR fans, and
V. Thermal Energy Savings is at least about 7327 BTU/gal of ethanol, or at least about 11,780 BTU/gal of Hydrocarbon (based on 100 MMGPY ETOH and 62.2 MMGPY of Hydrocarbon).

An added water stripper takes up the hydraulic load from the bottom of the rectifier column and reduces the hydraulic load on the beer stripper column. This reduces the size (diameter) of the beer stripper and enables the column to be fabricated in the shop and transported to the field. This provides significant cost savings over a field fabricated column.

Another way is to generate low pressure steam from an evaporator condensate or boiler water to drive an auxiliary reboiler for the water stripper that reduces the Natural Gas Thermal load by about 1,884 BTU/gal of ethanol, or about 3,028 BTU/gal of hydrocarbon.

The rectifier overhead vapor is partially condensed in the reboiler, so additional heat can be recovered by condensing more the rectifier vapor in the first of two effects in the finishing section of the evaporator system as shown in FIG. 12. FIG. 12 shows the evaporator scheme that integrates the rectifier column overheads in FIG. 11. FIG. 12 shows one configuration of the finishing evaporator that is useful to recover additional heat, but other configurations are within the spirit and scope of the present application.

FIG. 12 also shows thin stillage being concentrated into syrup. The MVR evaporator uses a single MVR fan to increase the water vapor pressure and temperature from the top of the evaporator to produce the heat neccesary to drive water off the thin stillage being fed to it. The discharge of the single MVR fan is used for the reflux of a distillation system.

The mid-stillage produced from the MVR evaporator is about 35 wt % to 45 wt % solids and is sent to a corn oil separation system where corn oil is removed via centrifugation. Optionally, the distiller corn oil generated by the corn oil separation may be consumed by the NZ plant as energy to reduce the plant's footprint. Alternatively, the distiller corn oil may be disposed of in other carbon reducing manners.

The mid-stillage is then sent to the finishing evaporator section where rectifier overhead vapor from the reboiler of the beer column is condensed and used to provide the heat for removing the rest of the water to achieve a syrup solids content of about 35 wt % tr 45 wt %.

Excess vapors from beer stripper reboiler are utilized in a 2-stage finisher (evaporator), which increase the syrup solids from at least about 35% to at least about 45%. This allows for better wet cake solids for shipping and sales, and reduces the energy in a rotary dryer by about 1.8 MMBTU/hr.

The condensed rectifier vapor is then sent back to the rectifier overhead tank where it can be used again for reflux.

Heat Integration in ethanol and hydrocarbon results in energy reduction of about 3,446 BTU/gal of ethanol, or about 5,540 BTU/gal of hydrocarbon.

Another feature apparent from FIGS. 11 and 12 is that the disclosed scheme improves efficiency because the beer stripper overheads is not condensed before feeding into the rectifier. With this feature, high rates of steam are not needed for the side stripper reboiler to re-vaporize the feed to the rectifier. The scheme in FIG. 11 incorporates the evaporator system into a heat-integration strategy. After the beer column reboiler, a portion of the rectifier overheads is still vapor. The condensing load of this remaining vapor is used to drive the finishing evaporator that is part of the evaporator system that produces syrup.

The scheme in FIG. 12 uses an MVR fan to drive an evaporator to remove a portion of the water in the thin stillage. The benefit of MVR fan is that it increases the overall efficiency of the evaporator and it eliminates the need for fresh steam in exchange for electricity from a renewable source like wind. The thin stillage feed to the evaporator system, as shown in FIG. 12, originates at the bottom of the beer column, as shown in FIG. 11, as whole stillage. The whole stillage is processed through a system to remove most of the insoluble solids (also known as suspended solids) from the liquid. Some type of centrifugation is typically employed for this. The two streams that come from the separation of whole stillage are referred to as thin stillage and wet cake. An example of the whole stillage, thin stillage, and wet cake flows and compositions are shown in Table 2. These correspond to over a 100 million gallon per year anhydrous ethanol capacity (about 8000 operating hours per year).

TABLE 2

| | Whole | Thins | |
|---|---|---|---|
| Component | Stillage | Stillage | Wet Cake |
| Large fibers + ash, | 16,482 | 2,472 | 14,010 |
| lb/hr | | | |
| Fine fibers + ash, | 12,034 | 4,212 | 7,822 |
| lb/hr | | | |
| Sugar (dextrose), | 778 | 597 | 181 |
| lb/hr | | | |
| Fine starch, lb/hr | 3,328 | 1,165 | 2,163 |
| Fine protein, lb/hr | 22,423 | 7,848 | 14,575 |
| Large protein, lb/hr | 0 | — | — |
| Corn glycerides, lb/hr | 10,606 | 6,576 | 4,030 |
| Corn FFA, lb/hr | 442 | 274 | 168 |
| Water, lb/hr | 354,949 | 272,477 | 82,472 |
| Lactic acid, lb/hr | 78 | 60 | 18 |
| Glycerol, lb/hr | 2,969 | 2,279 | 690 |
| Amylase protein, | 9 | 7 | 2 |
| lb/hr | | | |
| Yeast, lb/hr | 3176 | 476 | 2,700 |
| Urea, lb/hr | 134 | 103 | 31 |
| Total, lb/hr | 427,410 | 298,547 | 128,862 |

Often a portion of the thin stillage is recycled back to the slurry tank to mix with incoming corn flour, cook water, and enzymes. This recycled thin stillage is called backset. The backset serves multiple functions. It helps reduce fresh water usage to the plant and reduces the size and utilities associated with the evaporator system. It also provides nutrients that are utilized by the yeast during fermentation ensuring that the yeast are healthy and perform quickly and efficiently.

The diagram in FIG. 12 shows the thin stillage feeding into an MVR-powered evaporator. A constant circulation of thickened stillage is pumped from the bottom of the evaporator to the top. The thin stillage feed mixes with this recirculation flow as it travels to the top of the evaporator. This type of evaporator is referred to as a falling-film type of a evaporator because the feed to the top is distributed over an array of tubes. The stillage flows as a film down the inside of the tubes and is heated by the hot vapor flowing over the outside of the tubes. Water vapor evaporates from the thin stillage as it travels down the tubes and is allowed to disengage from the liquid at the bottom of the evaporator. In FIG. 12, the water vapor that separates from the stillage at the bottom is fed to an MVR fan which compresses the water vapor and forces it across the outside of the tubes where the majority of it condenses as it heats the stillage on the inside of the tubes. With the MVR, no fresh steam is needed to run this evaporator. The stillage stream produced in the MVR evaporator contains about 35 wt % to about 45 wt % total solids. This concentration is necessary to remove distillers' corn oil from the stillage stream in between the MVR evaporator and the finishing evaporators. The process for removing distillers' corn oil typically involves the addition of a chemical to the stillage that acts as a demulsifier followed by centrifugation. The corn oil, being less dense than water, can be separated quickly and efficiently under the high relative centrifugal forces imposed by centrifugation.

In the case of this lower energy process to produce biofuels, the distillers' corn oil is used as a fuel to run the boiler system.

The de-oiled mid-stillage from the oil separation block in FIG. 12 is sent to the finishing evaporators. The heat to drive the first finishing evaporator is supplied by the partially condensed rectifier overhead stream from the beer column reboiler. The stillage is circulated up to the top and back down through the tubes in the same manner as the MVR evaporator, except in this case the high proof rectifier vapor fed to the shell side of the first finisher evaporator condenses as it transfers heat through the tube walls into the stillage. The separated water vapor at the bottom of the first finisher evaporator is used to drive the second finisher evaporator. The concentrated stillage produced by the second finisher evaporator is referred to as syrup and is mixed with the wet cake from whole stillage separation. The wet cake can be sold as-is as animal feed and is referred to as wet distillers' grains with solubles (WDGS). The wet cake can also be dried and then sold as animal feed referred to as dry distillers' grains with solubles (DDGS). The water vapor from the second finishing evaporator is condensed and mixed with the other cook water streams to be used in the slurry tank.

The Table 3 below compares the energy usage for a conventional ethanol distillation and evaporation system, as shown in FIGS. 9 and 10 versus the system with energy reductions shown in FIGS. 11 and 12.

TABLE 3^{a}

| | Case 1^{b} | Case 2^{c} |
|---|---|---|
| Parameter | Conventional | Low Energy |
| MVR Fan Power, Megawatts | 0 | 6.4 |
| MVR Fan Power, MMBtu/hr | 0 | 21.8 |
| Steam Usage, MMBtu/hr | 117 | 15.8 |
| Natural Gas Equiv. MMBtu/hr^{d} | 137.6 | 18.6 |

^{a}All values based on a 100 million gallon per year anhydrous ethanol capacity and 8000 operating hours;
^{b}For FIGS. 9 and 10;
^{c}For FIGS. 11 and 12; and
^{d}Assumes a boiler efficiency of about 85%.

It is evident from Table 3, the steam demand drops dramatically when using the MVR based system by about 86.5%. In addition, when placing the MVR power requirement in the same units as the steam, it is not a one-to-one trade from steam to electricity. The MVR realizes an energy reduction of about 67% compared to the conventional independent of heat source.

In the disclosed lower energy process to produce biofuels, all electrical demands are satisfied with renewable power generated from wind. It is also feasible to use other forms of renewable electricity such as solar, nuclear or hydroelectric. For the disclosed lower energy process to produce biofuels, thermal loads that is normally be satisfied by natural gas can be satisfied with fuel streams generated in-situ by the process itself or from a blend of natural gas and renewable natural gas whose net carbon intensity is zero.

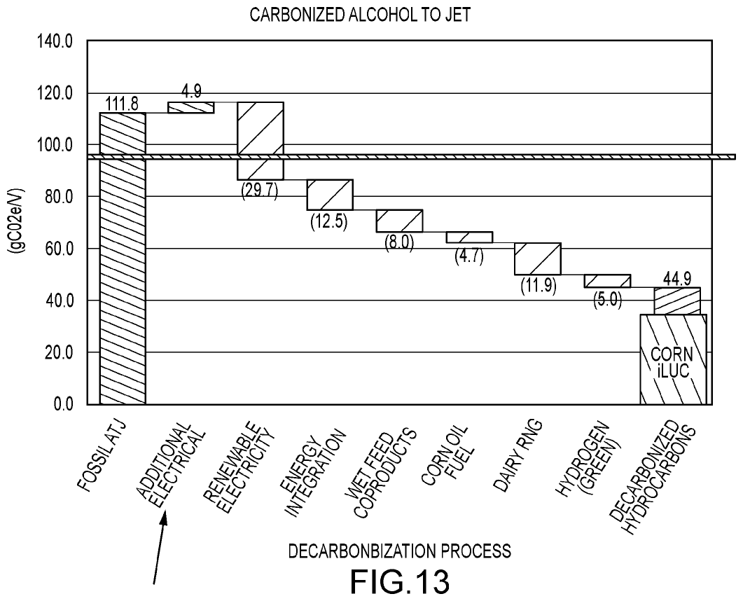

The disclosed low energy process to produce biofuels, for the jet fuel, naphtha, and renewable diesel products are marketable and valuable as renewable fuels, since their carbon intensity is low compared to their conventional petroleum-based counterparts. This low energy process to produce biofuels is designed to produce liquid transportation fuels possessing a carbon intensity of about 45 or less using the methodologies defined by the California Air Resource Board's (CARB) Low Carbon Fuel Standard (LCFS). FIG. 13 shows a comparison of carbon intensities for different petroleum-based fuels as well as the carbon intensity reductions by employing various aspects of the this low energy process to produce biofuels design to ultimately achieve a carbon intensity of about 45 or less for the liquid transportation fuel products.

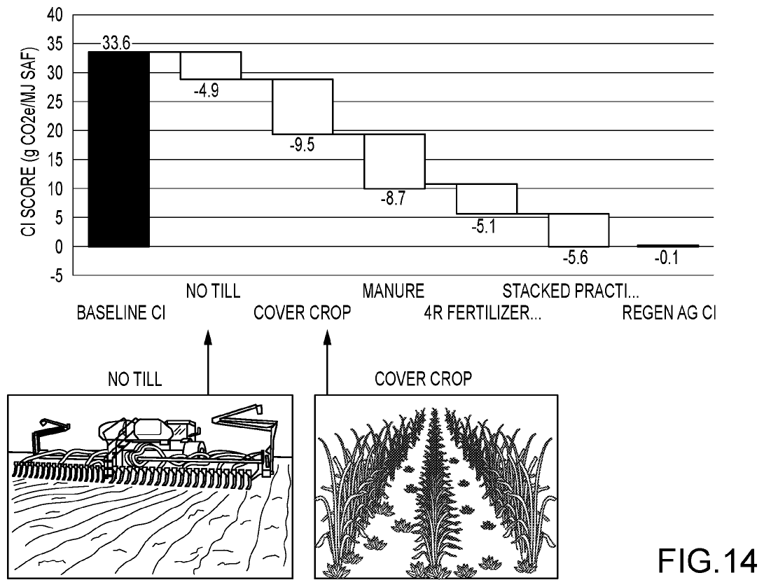

FIG. 14 exemplifies how low CI corn can be produced through climate smart agricultural practices. FIG. 14 illustrates the impact of various corn production agricultural practice on the CI of the sustainable aviation fuel (SAF) produced by the Gevo's system, which is modelled in Argonne GREET compared to a baseline of conventional tillage, with no cover crop, no manure, and no 4R fertilizers. It appears that more carbon can be captured in the soil. Sustainable agriculture offers potential upside in combination with renewable energy in production. Sustainable agriculture practices work synergistically to produce even better results than when used in isolation. It is evident from FIG. 14 that stacking practice improves soil health for greater soil organic carbon (SOC) and more resilient crops. Farmers Near Net-Zero 1 are doing sustainable agriculture and an average farm CI reduction near NZ1 is −6 g GHG/MJ SAF.

The specific examples presented herein are intended to be illustrative and should not be construed as limiting in scope of the claims.

The foregoing detailed description has been given for clearness of understanding only and no unnecessary limitations should be understood there from as modifications will be obvious to those skilled in the art.

While described in connection with specific embodiments thereof, it will be understood that the principles described herein is capable of further modifications and this application is intended to cover any variations, uses, or adaptations following, in general, the principles disclosed herein and including such departures from the present disclosure as come within known or customary practice within the art to which the technology pertains and as may be applied to the essential features hereinbefore set forth and as follows in the scope of the appended claims.

The disclosures, including the claims, figures and/or drawings, of each and every patent, patent application, and publication cited herein are hereby incorporated herein by reference in their entireties.

### EXEMPLARY ASPECTS/EMBODIMENTS

Certain aspects, including embodiments/aspects of the present subject matter described above, may be beneficial alone or in combination, with one or more other aspects recited hereinbelow. In addition, while the present subject matter has been disclosed with reference to certain aspects recited below and, in the claims, numerous modifications, alterations, and changes to the described aspects/embodiments are possible without departing from the sphere and scope of the present disclosure. Accordingly, it is intended that the present disclosure not be limited to the described embodiments, aspects, and claims, but that it has the full scope defined by the language of this disclosure and equivalents thereof. While the present technology has been described with reference to the specific aspects/embodiments thereof, it should be understood by those skilled in the art that various changes may be made and equivalents may be substituted without departing from the true spirit and scope of the disclosure. In addition, many modifications may be made to adapt a particular situation, material, composition of matter, process, and/or process step or steps, to the objective, spirit and scope of the present disclosure. All such modifications are intended to be within the scope of the aspects below appended hereto. The aspects below are all within the scope of the present application and may be claimed in whole or in part in the claims appended the present application.
1. A system for producing renewable hydrocarbons, comprising:
   a fractionation subsystem for processing a biomass that contains a carbohydrate;
   a fermentation subsystem for converting the carbohydrate to a fermentation product;
   an alcohol enrichment subsystem for receiving a second portion of the fermentation product;
   a hydrocarbon production subsystem for receiving a first portion of the fermentation product, which is an alcohol, and producing the renewable hydrocarbons;
   an energy management subsystem for receiving a fuel stream from the system and for delivering power to the system; and the energy management subsystem further comprising at least one of the following to reduce the energy demand or carbon intensity (CI) of the alcohol and/or fuel:
      a. a water treatment subsystem for receiving a first portion of the fermentation product;
      b. combustion of at least one of light oxygenate, fusel oil, vegetable oil, biomass or derivative, hydrocarbon co-products, and combinations thereof;
      c. using warm water sources and waste heat from the fermentation and hydrocarbon production subsystems for biomass processing;
      d. using a mechanical vapor recompression (MVR) system to pressurize rectifier column overheads, a condensate of which provides heat to a beer reboiler and produces a purified alcohol suitable for conversion to hydrocarbons; and
      e. a thin stillage evaporation system that use the heat from a partially condensed stream of 190 proof alcohol from the rectifier column overheads as well as the MVR system to produce a syrup that is at least about 35% to at least about 45% dry solids without any additional application of steam.
2. The system of aspect 1, wherein the fractionation subsystem further comprises at least one of:
   a storage vessel for storing the biomass;
   a pulverizer for breaking apart the biomass;
   a pretreatment vessel for pretreating the biomass;
   a treatment vessel for treating the biomass to produce the carbohydrate; and
   a high-temperature-short-time (HTST) vessel for pasteurizing the carbohydrate.
3. The system of aspect 2, wherein the biomass is corn.
4. The system of aspect 2, wherein the biomass has a negative carbon footprint.
5. The system of aspect 2, wherein the biomass grown using strip till or no till.
6. The system of aspect 2, wherein the pulverizer is a mill.
7. The system of aspect 2, wherein the pretreatment vessel includes a recycled water input.
8. The system of aspect 2, wherein pretreating the biomass includes pretreating the biomass with an enzyme.
9. The system of aspect 2, wherein the treatment vessel includes a non-fermentable solids output.
10. The system of aspect 9, wherein the non-fermentable solids output includes dried distillers grain output and/or corn oil output.
11. The system of any preceding aspect, wherein the fermentation subsystem comprises at least one of:
   a fermenter for converting the carbohydrate to the fermentation product;
   a nutrient addition subsystem;
   a pH adjustment subsystem; and
   an inoculum propagation subsystem.
12. The system of aspect 11, wherein the fermenter is a continuous fermenter.
13. The system of aspect 11, wherein the fermentation product includes an alcohol.
14. The system of aspect 13, wherein the alcohol is isobutanol.
15. The system of aspect 11, wherein the inoculum propagation subsystem is configured to provide a microorganism to the fermenter.
16. The system of aspect 15, wherein the microorganism is yeast.
17. The system of any preceding aspect, wherein the water treatment subsystem comprises at least one of:
   a beer well for receiving the first portion of the fermentation product;
   a microorganism separation device for removing microorganisms from the first portion of the fermentation product;
   a first distillation column for separating the first portion of the fermentation product into an alcohol and a bottom product;
   a digester for receiving a portion of the bottom product and for producing a biogas; and
   a biogas removal subsystem for removing a pollutant from the biogas.
18. The system of aspect 17, wherein the first portion of the fermentation product includes an alcohol.
19. The system of aspect 17, wherein the first portion of the fermentation product includes isobutanol.
20. The system of aspect 17, wherein the first portion of the fermentation product includes water.
21. The system of aspect 17, wherein the microorganism separation device includes a centrifuge.
22. The system of aspect 17, wherein the microorganism separation device includes a filter.
23. The system of aspect 17, wherein the microorganism separation device includes a settling tank.
24. The system of aspect 17, wherein the microorganisms are yeast.
25. The system of aspect 17, wherein the first distillation column include vapor recompression subsystem.
26. The system of aspect 17, wherein the first distillation column include mechanical vapor recompression (MVR).
27. The system of aspect 17, wherein the first distillation column include thermal vapor recompression (TVR).
28. The system of aspect 17, wherein the bottom product includes stillage.
29. The system of aspect 17, wherein the digester is an anaerobic digester.
30. The system of aspect 17, wherein the digester is a continuous digester.
31. The system of aspect 17, wherein the biogas removal subsystem includes a scrubber.
32. The system of aspect 17, wherein the pollutant is hydrogen sulfide.
33. The system of any preceding aspect, wherein the alcohol enrichment subsystem comprises at least one of:
   a flash tank for separating an condensate from the second portion of the fermentation product;
   a separation vessel for separating the condensate into a light phase and a heavy phase;
   an ion exchange vessel for purifying the light phase;
   a membrane separator for separating the light phase into an alcohol-high retentate and a water-rich permeate;
   a second distillation column for separating water from the alcohol-high retentate; and
   an alcohol storage vessel.
34. The system of aspect 33, wherein the flash tank receives the second portion of the fermentation product from the fermentation subsystem and returns back part of the second portion of the fermentation product to the fermentation subsystem.
35. The system of aspect 33, wherein the second portion of the fermentation product is a broth and an isobutanol lean broth is returned back to the fermentation subsystem.
36. The system of aspect 33, wherein the flash tank operates at atmospheric pressure.
37. The system of aspect 33, wherein the flash tank operates under a vacuum at a reduced pressure below atmospheric pressure.
38. The system of aspect 33, wherein the flash tank operates at a temperature below a temperature of the second portion of the fermentation product received from the fermentation subsystem.
39. The system of aspect 33, wherein the separation vessel is a liquid/liquid separator for separating the condensate into the light phase and the heavy phase.
40. The system of aspect 39, wherein the light phase contains a greater amount of alcohol than the heavy phase.
41. The system of aspect 40, wherein the alcohol is isobutanol and the liquid/liquid separator form a biphasic system.
42. The system of aspect 33, wherein the ion exchange vessel contains an ion exchange resin and receives the light phase received from the separation vessel and removes impurities from the light phase by trapping ions in the ion exchange resin.
43. The system of aspect 42, wherein the ion exchange vessel receives a caustic solution to regenerate the ion exchange resin by flushing the impurities to a beer well.
44. The system of aspect 33, wherein the hydrocarbon production subsystem comprises at least one of:
   a denitrogination subsystem for separating nitrogen from the alcohol;
   a dehydration subsystem for converting the alcohol to an olefin;
   a hydrocarbon pretreatment subsystem for conditioning an olefin feed;
   a hydrogen supply subsystem for proving hydrogen; and
   a hydrocarbon processing subsystem for converting the olefin feed to at least one of an iso-octane fraction, a C₁₂ alkane fraction, and/or C₁₆ alkane fraction.
45. The system of aspect 44, wherein the hydrocarbon production subsystem further comprises at least one of:
   an oil and water separator for separating wastewater from the hydrocarbon processing subsystem; and
   a hydrocarbon storage vessel for storing at least one of iso-octane, C₁₂ blend, or C₁₆ blend from the hydrocarbon processing subsystem.
46. The system of aspect 44, wherein the denitrogination subsystem receives the alcohol from the alcohol enrichment subsystem.
47. The system of aspect 46, wherein the alcohol is a fuel grade isobutanol.
48. The system of aspect 44, wherein the dehydration subsystem receives the alcohol from the denitrogination subsystem and sends water and/or purge products to a beer well.
49. The system of aspect 44, wherein the olefin is a four carbon olefin.
50. The system of aspect 44, wherein the hydrocarbon pretreatment subsystem comprises at least one of:
   a coalescer for receiving the olefin from the dehydration subsystem;
   an adsorber for removing unreacted alcohols, water, and nitrogen; and
   a feed receiver.
51. The system of aspect 50, wherein the coalescer further receives wash water and sends waste water to a surge tank and/or a dehydration unit.
52. The system of aspect 50, wherein the coalescer further receives wash water and sends waste water to a surge tank and/or a dehydration unit.
53. The system of aspect 50, wherein the olefin is a C₄ olefin.
54. The system of aspect 50, wherein the unreacted alcohol is isobutanol.
55. The system of aspect 50, wherein the feed receiver receives the olefin from the adsorber.
56. The system of aspect 44, wherein the hydrocarbon processing subsystem comprises at least one of:
   a first reactor;
   a debutanizer;
   a second reactor;
   a separator;
   a first splitter; and/or
   a second splitter.
57. The system of aspect 56, wherein the first reactor is a polynaptha reactor.
58. The system of aspect 56, wherein the first reactor receives the olefin from the feed receiver and oligomerizes the olefin to a polyolefin.
59. The system of aspect 56, wherein the debutanizer receives a polyolefin from the first reactor.
60. The system of aspect 56, wherein the debutanizer recycles an unreacted olefin and/or paraffin to the first reactor.
61. The system of aspect 56, wherein the second reactor is a hydrogenation reactor.
62. The system of aspect 56, wherein the second reactor receives a polyolefin from the debutanizer.
63. The system of aspect 56, wherein the second reactor further receives hydrogen from a hydrogen supply subsystem and hydrogenates the polyolefin to a C₈-C₁₆ alkane.
64. The system of aspect 56, wherein the separator removes an unreacted olefin and/or paraffin from the C₈-C₁₆ alkane.
65. The system of aspect 64, wherein the unreacted olefins and/or paraffins include C₄ products.
66. The system of aspect 56, wherein the separator recycles a portion of the C₈-C₁₆ alkane to the second reactor.
67. The system of aspect 56, wherein the first splitter separates the isooctane fraction from the C₈-C₁₆ alkane.
68. The system of aspect 67, wherein the isooctane fraction comprises about 85%, 90%, 91%, 92%, 93%, 94%, 95, 96%, 97%, 98%, 99% or 100% wt % C₈ alkanes, about 1%, 2%, 3%, 4% or 5 vol % olefins, and/or about 2 ppm, 4 ppm, 6 ppm, 8 ppm or 10 ppm sulfur.
69. The system of aspect 67, wherein the isooctane fraction comprises greater than 95 wt % C8 alkanes, less than 5 vol % olefins, and/or less than 10 ppm sulfur.
70. The system of aspect 56, wherein the first splitter separates an unreacted olefin and/or paraffin from the C₈-C₁₆ alkane.
71. The system of aspect 70, wherein the unreacted olefins and/or paraffins include C₄ products.
72. The system of aspect 56, wherein the second splitter separates the C₁₂ alkane fraction from the C₁₈ fraction.
73. The system of any preceding aspect, wherein the energy management subsystem comprises at least one of:
   a fuel gas system for distributing fuel gas received from the hydrocarbon processing subsystem;
   a low pressure boiler for generating steam;
   a high pressure boiler for generating steam;
   a combined heat and power unit for generating steam and electricity;
   a wind turbine for generating electricity;
   an electric boiler for receiving renewable electricity and for heating water to produce steam;
   a biomass boiler for combusting biomass for heating water to produce steam; and/or
   a steam turbine for generating electricity.
74. The system of aspect 73, wherein the fuel gas system provides fuel gas to the low pressure boiler.
75. The system of aspect 73, wherein the fuel gas system provides fuel gas to the high pressure boiler.
76. The system of aspect 73, wherein the fuel gas system provides fuel gas to a fired heater.
77. The system of aspect 73, wherein the low pressure boiler is further configured to received natural gas and/or biogas from an anaerobic digester.
78. The system of aspect 73, wherein the combined heat and power unit is further configured to received natural gas and/or biogas from an anaerobic digester.
79. A system for producing renewable alcohols, comprising:
   a fractionation subsystem for processing a biomass that contains a carbohydrate;
   a fermentation subsystem for converting the carbohydrate to a fermentation product;
   an alcohol enrichment subsystem for receiving at least a portion of the fermentation product;
   an energy management subsystem for receiving fuel stream from the system and for delivering power to the system; and at least one of the following to reduce the energy demand or CI of the alcohol:
      a) a water treatment subsystem for receiving a first portion of the fermentation product;
      b) combustion of light oxygenate, fusel oil, vegetable oil, biomass or derivative, and/or co-products;
      c) utilizing an alcohol purification system that uses a mechanical vapor recompression system to pressurize the rectifier column overheads, the condensation of which provides heat to the beer stripper reboiler; and
      d) a thin stillage evaporation system that utilizes the heat from a partially condensed stream of 190 proof ethanol from the rectifier overheads as well as a mechanical vapor recompression system to produce a syrup that is at least about 35% to at least about 45% dry solids without any additional application of steam.

What is claimed is:

1. A system for producing renewable hydrocarbons, comprising:
a fractionation subsystem for processing a biomass that contains a carbohydrate;
a fermentation subsystem for converting the carbohydrate to a fermentation product;
an alcohol enrichment subsystem for receiving a second portion of the fermentation product;
a hydrocarbon production subsystem for receiving a first portion of the fermentation product, which is an alcohol, and producing the renewable hydrocarbons;
an energy management subsystem for receiving a fuel stream from the system and for delivering power to the system; and the energy management subsystem further comprising at least one of the following to reduce the energy demand or carbon intensity (CI) of the system:
a. a water treatment subsystem for receiving a part of the first portion of the fermentation product;
b. combustion of at least one of light oxygenate, fusel oil, vegetable oil, biomass or derivative, hydrocarbon co-products, and combinations thereof;
c. using warm water sources and waste heat from the fermentation and hydrocarbon production subsystems for biomass processing;
d. using a mechanical vapor recompression (MVR) system to pressurize rectifier column overheads, a condensate of which provides heat to a beer reboiler and produces a purified alcohol suitable for conversion to hydrocarbons; and
e. a thin stillage evaporation system that use the heat from a partially condensed stream of 190 proof alcohol from the rectifier column overheads as well as the MVR system to produce a syrup that is at least about 35% to at least about 45% dry solids without any additional application of steam.

2. The system of claim 1, wherein the fractionation subsystem further comprises at least one of:
a storage vessel for storing the biomass;
a pulverizer for breaking apart the biomass;
a pretreatment vessel for pretreating the biomass;
a treatment vessel for treating the biomass to produce the carbohydrate; and
a high-temperature-short-time (HTST) vessel for sterilizing the carbohydrate.

3. The system of claim 1, wherein the fermentation subsystem comprises at least one of:
a fermenter for converting the carbohydrate to the fermentation product;
a nutrient addition subsystem;
a pH adjustment subsystem; and
an inoculum propagation subsystem.

4. The system of claim 3, wherein the fermenter is a continuous fermenter.

5. The system of claim 3, wherein the inoculum propagation subsystem is configured to provide a microorganism to the fermenter.

6. The system of claim 1, wherein the water treatment subsystem comprises at least one of:
a beer well for receiving the first portion of the fermentation product;
a microorganism separation device for removing microorganisms from the first portion of the fermentation product;
a first distillation column for separating the first portion of the fermentation product into an alcohol and a bottom product;
a digester for receiving a portion of the bottom product and for producing a biogas; and
a biogas removal subsystem for removing a pollutant from the biogas.

7. The system of claim 6, wherein the first distillation column includes a vapor recompression system.

8. The system of claim 7, wherein the first distillation column include mechanical vapor recompression (MVR).

9. The system of claim 7, wherein the first distillation column include thermal vapor recompression (TVR).

10. The system of claim 1, wherein the alcohol enrichment subsystem comprises at least one of:
a flash tank for separating a condensate from the second portion of the fermentation product;
a separation vessel for separating the condensate into a light phase and a heavy phase;
an ion exchange vessel for purifying the light phase;
a membrane separator for separating the light phase into an alcohol-high retentate and a water-rich permeate;
a second distillation column for separating water from the alcohol-high retentate; and
an alcohol storage vessel.

11. The system of claim 10, wherein the flash tank receives the second portion of the fermentation product from the fermentation subsystem and returns back part of the second portion of the fermentation product to the fermentation subsystem.

12. The system of claim 1, wherein the hydrocarbon production subsystem comprises at least one of:
a denitrogination subsystem for separating nitrogen from the alcohol;
a dehydration subsystem for converting the alcohol to an olefin;
a hydrocarbon pretreatment subsystem for conditioning an olefin feed;
a hydrogen supply subsystem for proving hydrogen; and
a hydrocarbon processing subsystem for converting the olefin feed to at least one of an iso-octane fraction, a C₁₂ alkane fraction, and/or C₁₆ alkane fraction.

13. The system of claim 12, wherein the hydrocarbon production subsystem further comprises at least one of:
an oil and water separator for separating wastewater from the hydrocarbon processing subsystem; and
a hydrocarbon storage vessel for storing at least one of iso-octane, C₁₂ blend, or C₁₆ blend from the hydrocarbon processing subsystem.

14. The system of claim 12, wherein the hydrocarbon processing subsystem comprises at least one of:
a first reactor;
a debutanizer;
a second reactor;
a separator;
a first splitter; and
a second splitter.

15. The system of claim 1, wherein the energy management subsystem comprises at least one of:
a fuel gas system for distributing fuel gas received from the hydrocarbon processing subsystem;
a low pressure boiler for generating steam;
a high pressure boiler for generating steam;
a combined heat and power unit for generating steam and electricity;
a wind turbine for generating electricity;
an electric boiler for receiving renewable electricity and for heating water to produce steam;
a biomass boiler for combusting biomass for heating water to produce steam; and
a steam turbine for generating electricity.
